(19) United States
(12) Patent Application Publication
(10) Pub. No.: US 2025/0248801 A1
(43) Pub. Date: Aug. 7, 2025

(54) METHODS AND SYSTEMS FOR PACKAGING AND PREPARING A PROSTHETIC VALVE DELIVERY SYSTEM

(71) Applicant: EDWARDS LIFESCIENCES CORPORATION, Irvine, CA (US)

(72) Inventors: Vipul P. Rajpara, Lake Forest, CA (US); Martha Getaneh, Mission Viejo, CA (US); Michael R. Bialas, Lake Forest, CA (US); Ilan Tamir, Irvine, CA (US); Jason Seng-Che Lam, Lake Forest, CA (US); Quang Ngoc Vu, Aliso Viejo, CA (US)

(73) Assignee: EDWARDS LIFESCIENCES CORPORATION, Irvine, CA (US)

(21) Appl. No.: 19/190,449

(22) Filed: Apr. 25, 2025

Related U.S. Application Data
(63) Continuation of application No. PCT/US2023/079601, filed on Nov. 14, 2023.
(60) Provisional application No. 63/384,271, filed on Nov. 18, 2022.

(51) Int. Cl. A61F 2/00; A61B 50/30; A61B 50/33; A61F 2/24

(52) U.S. Cl. CPC A61F 2/0095; A61B 50/33; A61F 2/2433; A61B 2050/3005

(57) ABSTRACT

Packaging assemblies for a prosthetic valve delivery apparatus are described herein. As an example, an assembly can include a packaging tray comprising a plurality of recesses configured to receive components of a prosthetic valve delivery system, the tray having a first end portion, a second end portion, and a middle portion extending from the first end portion to the second end portion. The assembly includes a delivery apparatus disposed within a set of one or more first recesses of the tray such that a shaft of the delivery apparatus has a curved section forming a 180-degree bend along a distal half of the shaft, the curved section positioned within the first end portion, and a crimping device configured to be received within a second recess of the tray that is positioned within the middle portion, where a valve mounting portion of the delivery apparatus is adjacent the crimping device.

### CROSS-REFERENCE TO RELATED APPLICATIONS

This application is a continuation of PCT Application No. PCT/US2023/079601, filed Nov. 14, 2023, which claims the benefit of U.S. Provisional Patent Application No. 63/384,271, filed Nov. 18, 2022, the entire contents of each of which are incorporated by reference herein.

### FIELD

The present disclosure relates to delivery systems for expandable prosthetic heart valves and methods and systems for packaging and preparing such delivery systems.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (eg., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A delivery system for implanting a prosthetic valve can include the delivery apparatus, a prosthetic valve, and one or more additional components that aid in preparing and mounting the prosthetic valve onto the delivery apparatus and inserting the delivery apparatus into a patient. For example, a prosthetic valve delivery system can further include a crimping device used to crimp the prosthetic valve onto the delivery apparatus, a sheath used to insert the delivery apparatus into a patient, a loader used to load a distal end portion of the delivery apparatus that includes the prosthetic heart valve crimped thereon into the sheath, and/or the like. In some examples, one or more of the delivery system components can be packaged (for shipping and/or storage) separately into one or more packages. Upon receipt at an implantation facility, the packages can be opened, and the delivery system can be assembled and prepared for an implantation procedure. The preparation procedure can include soaking the prosthetic valve, flushing or de-airing the delivery apparatus, and then crimping the prosthetic valve onto the distal end portion of the delivery apparatus.

### SUMMARY

Described herein are prosthetic heart valves, delivery apparatuses and systems, a packaging for one or more components of the delivery systems, and methods for preparing a delivery apparatus and prosthetic heart valve for implantation in a patient. A delivery system for a prosthetic heart valve can comprise the prosthetic heart valve, a delivery apparatus, and a crimping device. In some examples, the delivery system can further include one or more alignment devices for aligning the prosthetic heart valve on the delivery apparatus, a sheath for inserting the delivery apparatus into a blood vessel of a patient, a loader for loading the distal end portion of the delivery apparatus including the prosthetic heart valve crimped thereon into the introducer sheath, and/or a syringe for de-airing the delivery apparatus. The delivery apparatus and one or more additional components for the valve and delivery apparatus preparation procedure (e.g., the loader and a fluid bowl) can be inserted into a tray of a packaging for shipping and/or storage. Upon receipt at an implantation facility (e.g., a procedure or operating room), the delivery apparatus packaging and one or more packagings holding additional components of the delivery system (e.g., the prosthetic heart valve and the crimping device) can be opened. The tray of the delivery apparatus packaging can be used as an integrated prep tray for preparing both the prosthetic heart valve and delivery apparatus for insertion into a patient. For example, the prosthetic heart valve can be soaked and crimped onto a distal end portion of the delivery apparatus all within the prep tray of the packaging. The disclosed prosthetic heart valves, delivery apparatuses, packaging, and methods can, for example, provide a way to prepare the prosthetic valve and delivery apparatus for a valve implantation procedure within a standardized prep tray of the packing, thereby simplifying the preparation procedure. As such, the devices and methods disclosed herein can, among other things, overcome one or more of the deficiencies of typical prosthetic heart valves and their delivery apparatuses.

An assembly can comprise a tray configured to receive a prosthetic valve delivery apparatus.

An assembly can comprise a packaging tray, a delivery apparatus for a prosthetic valve, and a crimping device, wherein the packaging tray comprises recesses for the delivery apparatus and the crimping device.

In some examples, an assembly can comprise a packaging tray, a delivery apparatus for a prosthetic valve, and a crimping device, wherein the packaging tray comprises a first space for the delivery apparatus and a second space disposed at a middle portion of the tray for the crimping device, wherein a curved section a shaft of the delivery apparatus forms a 180-degree bend, and wherein a valve mounting portion of the delivery apparatus is adjacent the crimping device when the crimping device is arranged within the tray.

In some examples, an assembly can comprise a tray comprising a plurality of recesses, a delivery apparatus disposed within a set of one or more first recesses of the plurality of recesses of the tray such that a handle portion of the delivery apparatus is positioned within a first end portion of the tray, a distal half of a shaft of the delivery apparatus has a curved section is positioned within a second end portion of the tray, and a valve mounting portion of the delivery apparatus is positioned within the tray closer to the second end than the first end.

In some examples, an assembly comprises a packaging tray comprising a plurality of recesses configured to receive components of a prosthetic valve delivery system, where the tray has a length, a first end portion extending along a third of the length, a second end portion, opposite the first end portion, extending along a third of the length, and a middle portion extending along a third of the length from the first end portion to the second end portion. The assembly further comprises a delivery apparatus disposed within a set of one or more first recesses of the plurality of recesses of the tray such that a shaft of the delivery apparatus has a curved section forming a 180-degree bend along a distal half of the shaft, where the curved section is positioned within the first end portion of the tray. The assembly further comprises a crimping device configured to be received within a second recess of the plurality of recesses that is positioned within the middle portion of the tray, where a valve mounting portion of the delivery apparatus is adjacent the crimping device when the crimping device is positioned within the second recess.

In some examples, a packaging assembly for storing a delivery system for a prosthetic valve comprises a tray comprising a plurality of recesses, where the tray comprises a first end portion and a second end portion opposite the first end portion, the first end portion defining a first end of the tray and the second end portion defining a second end of the tray. The packaging assembly further comprises a delivery apparatus disposed within a set of one or more first recesses of the plurality of recesses of the tray such that a handle portion of the delivery apparatus is positioned within the first end portion of the tray, a distal half of a shaft of the delivery apparatus has a curved section forming a 180-degree bend positioned within the second end portion of the tray, and a valve mounting portion of the delivery apparatus is positioned within the tray closer to the second end than the first end.

In some examples, a packaging assembly for storing a prosthetic valve delivery apparatus comprises a tray configured to receive the prosthetic valve delivery apparatus, where the tray includes at least one recess, and a removable bowl disposed in the recess, where the bowl is configured to receive a liquid.

In some examples, an assembly comprises one or more of the components recited in Examples 1-31 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic heart valve, according to one example.

FIG. 1B is a perspective view of the prosthetic valve of FIG. 1A with the components on the outside of the frame shown in transparent lines for purpose of illustration.

FIG. 2 is a side view of an example of a delivery apparatus configured to deliver and implant a radially expandable prosthetic heart valve at an implantation site.

FIG. 3 is a side view of an example of a sheath or introducer sheath used to insert a delivery apparatus into a blood vessel of a patient.

FIG. 4 is a side view of an example of a loader used to load a distal end portion of a delivery apparatus into an introducer sheath.

FIG. 5 is a perspective view of an example of a syringe used to de-air a delivery apparatus.

FIG. 6 is a perspective view of an example of a valve holder for a prosthetic valve.

FIG. 7 is a perspective view of an example of a positioning device for a delivery apparatus for a prosthetic valve.

FIG. 8 is a side view of the positioning device of FIG. 7 coupled to a distal end portion of a delivery apparatus.

FIG. 9 is a perspective view of an example of a crimping device.

FIG. 10 is an exploded view of a package including a packaging tray configured to receive and retain a delivery apparatus for a prosthetic valve therein.

FIG. 11 is a top view of the tray configured to be included in the package of FIG. 10.

FIG. 12 is a perspective view of the tray of FIG. 11.

FIG. 13 is a top view of the tray of FIG. 11 depicting a delivery apparatus and crimping device coupled to the tray.

FIG. 14 is a perspective view of the tray of FIG. 11 depicting the delivery apparatus and crimping device coupled to the tray.

FIG. 15 is a detail view of a portion of the tray of FIG. 11 which includes a recess that is configured to receive a crimping device.

FIG. 16 is a detail view of a portion of the tray of FIG. 11 showing a portion of a recess configured to receive a crimping device and a portion of a recess configured to receive a shaft of a delivery apparatus.

FIG. 17 is a detail view of a portion of the tray of FIG. 11 showing a portion of a recess configured to receive a shaft of the delivery apparatus and retain the shaft therein with pairs of opposing retaining features.

FIG. 18 is a perspective view of a first fluid bowl configured to be removably coupled to the tray of FIG. 11.

FIG. 19 is a perspective view of a second fluid bowl configured to be removably coupled to the tray of FIG. 11, the second fluid bowl including a bowl portion and holder portion that are foldable relative to one another.

FIG. 20 is a perspective view of a package for a prosthetic valve.

FIG. 21 is a top perspective view of the package of FIG. 20.

FIG. 22 is a perspective view of a retainer of the package of FIG. 10, the retainer configured to fit over the tray.

FIGS. 23A-23F are top views of the packaging of FIG. 11 depicting various stages in an exemplary method for using the tray of FIG. 11 as an integrated prep tray for a prosthetic valve and delivery apparatus preparation procedure.

FIG. 23G is perspective view of an exemplary valve holder and valve used in the method shown in FIGS. 23C-23F.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, a delivery apparatus for a prosthetic heart valve can be packaged for delivery within a packaging. In some examples, an entire delivery system for preparation and implantation of a prosthetic heart valve includes multiple components that can be packaged separately and then prepared and assembled on a table of an operating or procedure room, prior to the valve implantation procedure. In some examples, the delivery system can include a delivery apparatus which comprises a handle and a shaft extending from the handle, a prosthetic heart valve that is mountable around the distal end portion of the delivery apparatus shaft, and a crimper or crimping device configured to radially compress the prosthetic heart valve around the distal end portion of the delivery apparatus. In some instances, the delivery system can also include an introducer sheath, loader, and/or syringe which are packaged separately from the delivery apparatus.

The preparation procedure for the prosthetic heart valve and delivery apparatus can include soaking the prosthetic heart valve in a saline solution, flushing or de-airing the delivery apparatus with the syringe, and then crimping the prosthetic valve onto the distal end portion of the delivery apparatus (using the crimper). The separately packaged components of the delivery system can be prepared and assembled together on a sterile table in an operating room. However, different implantation facilities may perform the preparation procedure in different ways, thereby leading to inconsistencies in the procedure. Further, it can be difficult to maintain the delivery system components in place without them sliding around or off the table during the prep procedure (such as during crimping, which results in them being placed outside the sterile environment of the table).

Described herein are various methods and assemblies for packaging and preparing a prosthetic valve delivery system for an implantation procedure. For example, a packaging can comprise a packaging tray configured to receive a delivery apparatus therein for shipping and/or storage and the delivery apparatus can be retained in a specified orientation within the tray during a preparation procedure. The tray can further comprise a space or recess configured to receive a crimping device therein during the preparation procedure (e.g., in order to crimp a prosthetic valve onto the delivery apparatus within the tray). The space for the crimping device can be disposed in a middle portion of the tray. The shaft of the delivery apparatus can bend at approximately 180 degrees within the tray such that a distal end portion of the shaft which includes a valve mounting portion configured to receive the prosthetic valve thereon is arranged adjacent a valve-receiving portion of the crimping device. In this way, the delivery apparatus can be held within the tray during the preparation procedure and the prosthetic valve can be prepared and crimped onto the valve mounting portion of the delivery apparatus while the system components remain within the tray (which may be sterile). As a result, the preparation process can be performed in a contained and sterile environment and the preparation process can be simplified and standardized.

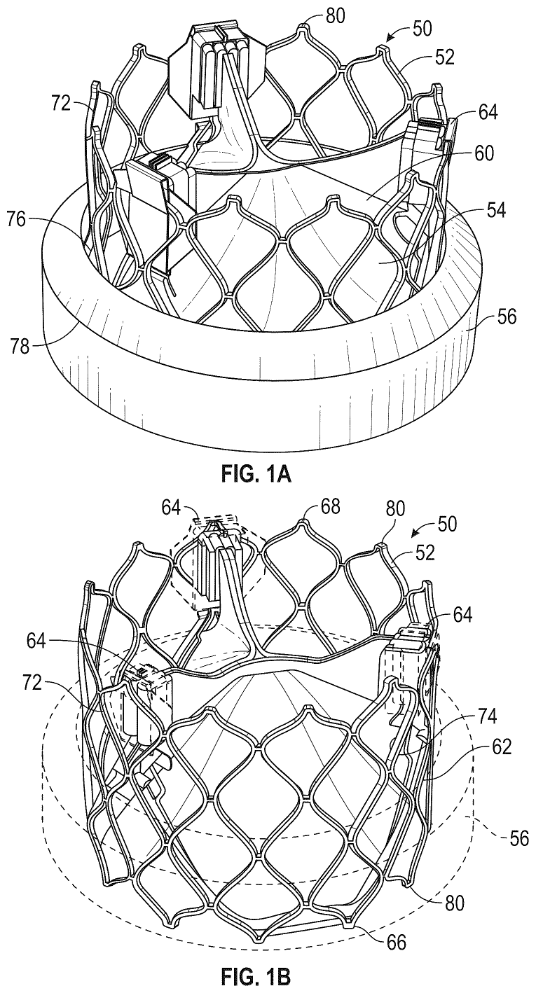

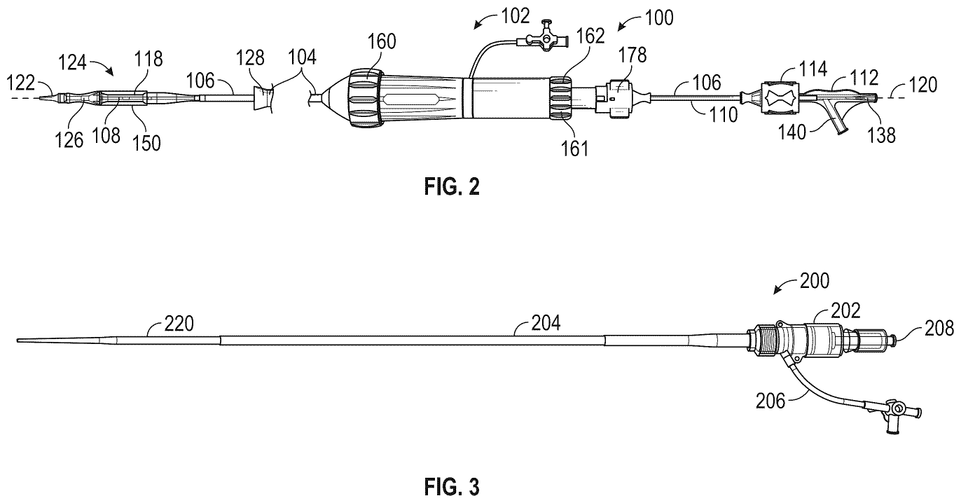

An exemplary prosthetic heart valve is shown in FIGS. 1A-1B. As introduced above, the prosthetic heart valve can be crimped on a delivery apparatus, such as the exemplary delivery apparatus shown in FIG. 2, in a radially compressed state while being advanced through a patient's vasculature on the delivery apparatus.

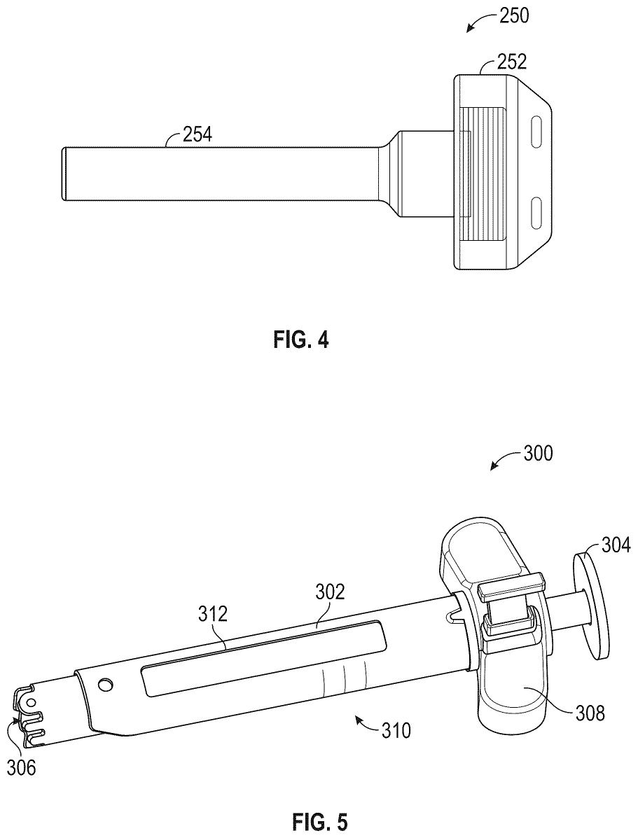

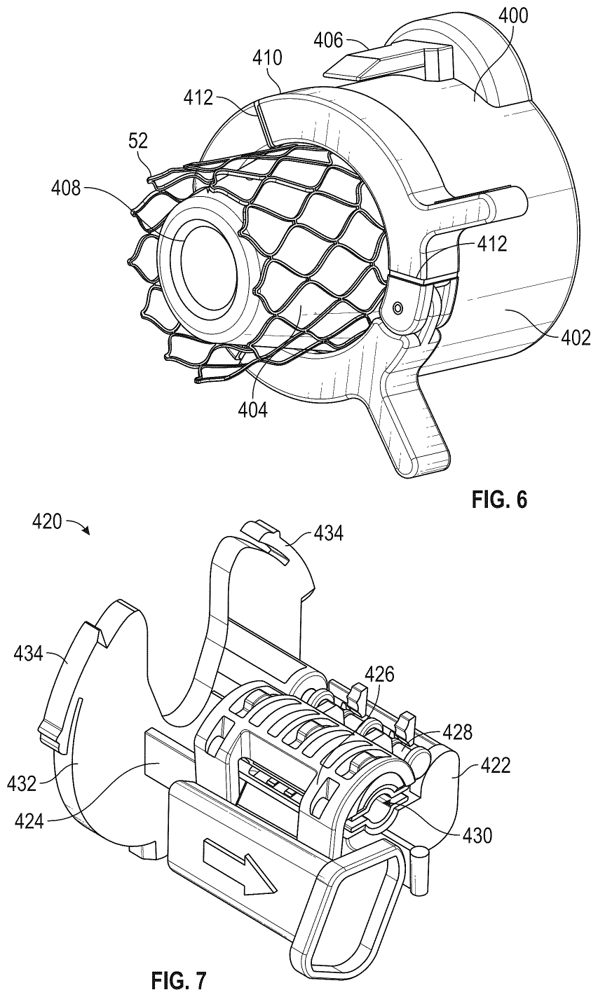

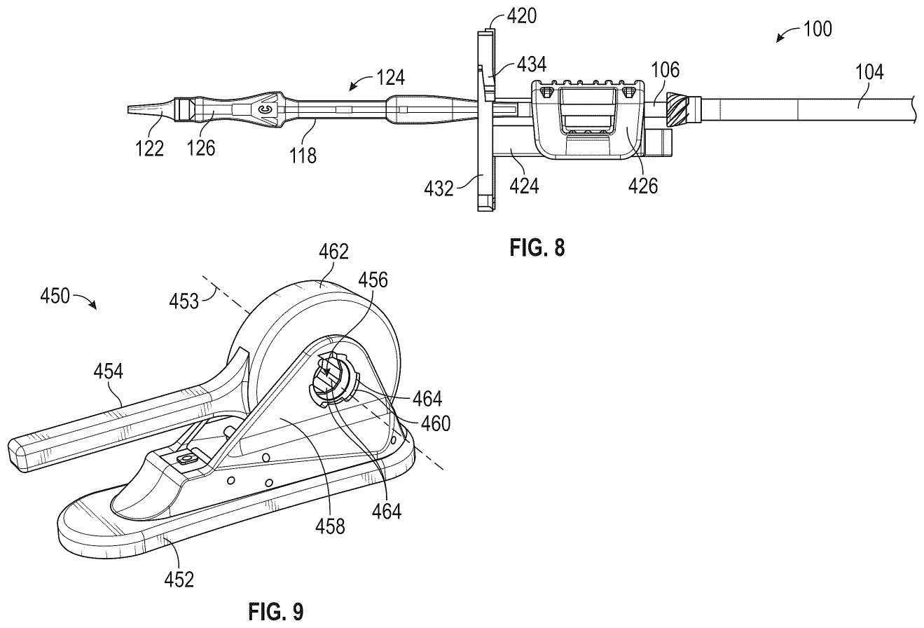

Exemplary components of a prosthetic valve delivery system that can be used during a preparation procedure for preparing a prosthetic heart valve and delivery apparatus for implantation into a patient can include a loader (FIG. 4), syringe (FIG. 5), valve holder (FIG. 6), positioning device (FIGS. 7 and 8), and a crimping device (FIG. 9). FIG. 3 shows an exemplary introducer sheath that can be used to insert the prepared and assembled prosthetic valve and delivery apparatus into a blood vessel of a patient.

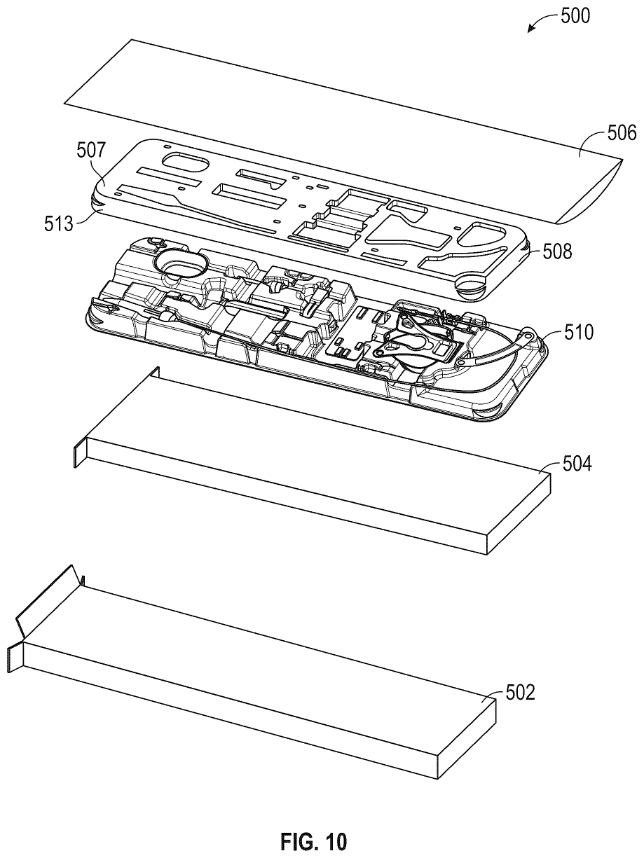

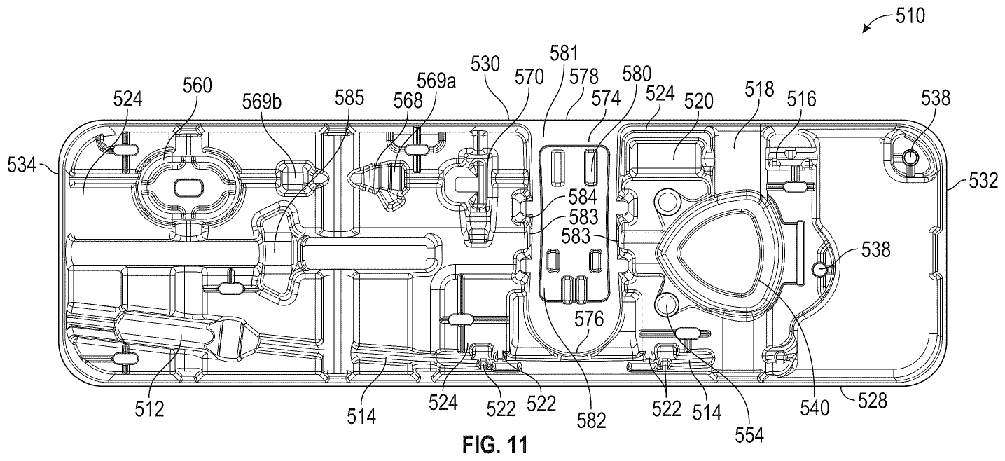

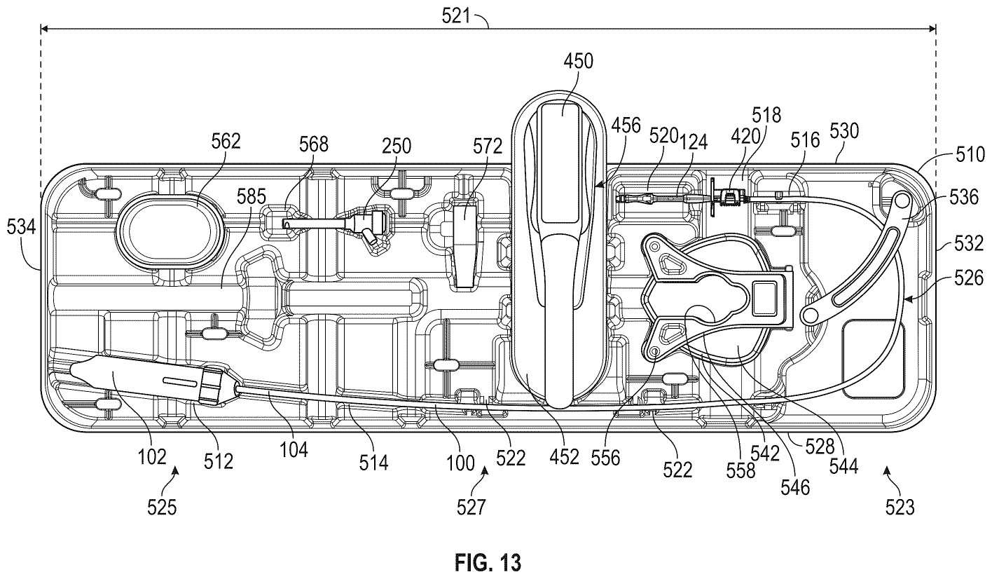

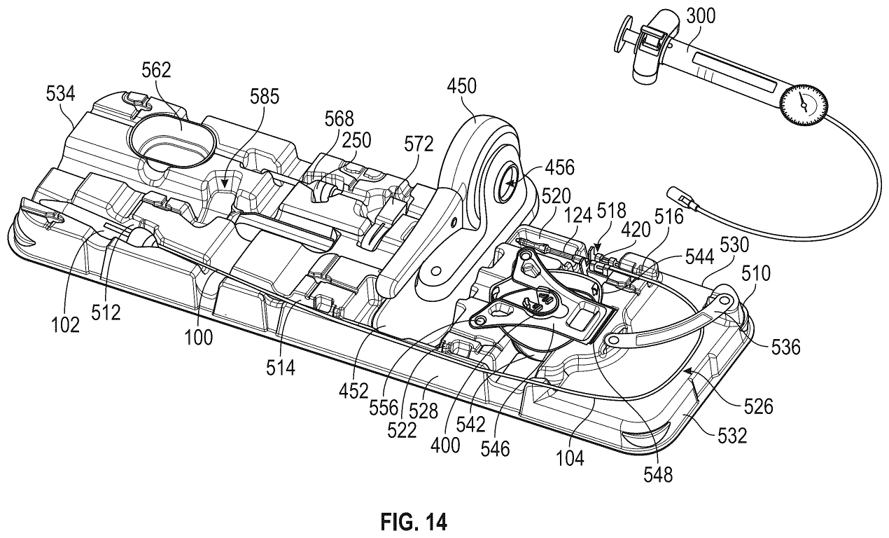

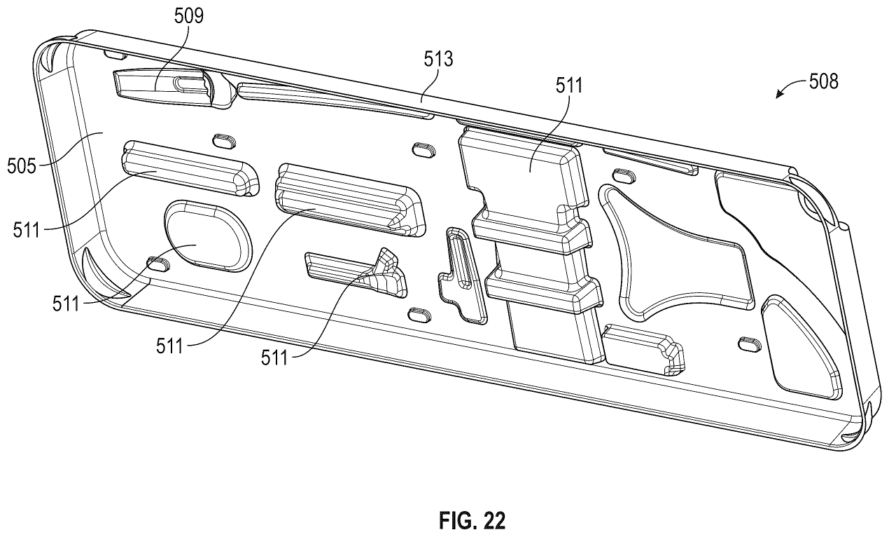

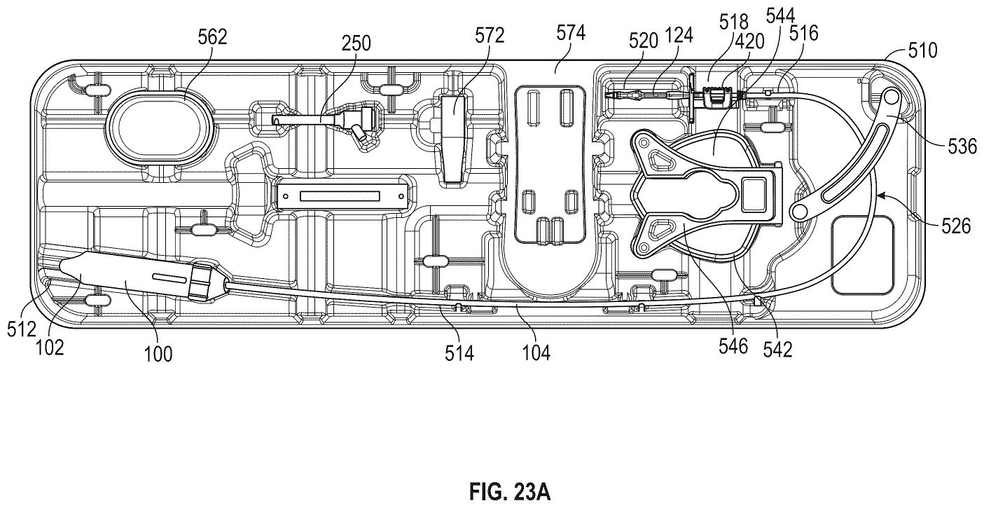

FIG. 10 shows an exemplary package that can be used to ship and store components of a prosthetic valve delivery system, such as the delivery apparatus. The package can comprise a tray comprising recesses (FIGS. 11, 12, and 15-17) that are configured to receive various components therein, such as the delivery apparatus and crimping device (FIGS. 13 and 14). The package can also comprise a retainer (FIG. 22) that is configured to fit over the tray for shipping, transport, and/or storage.

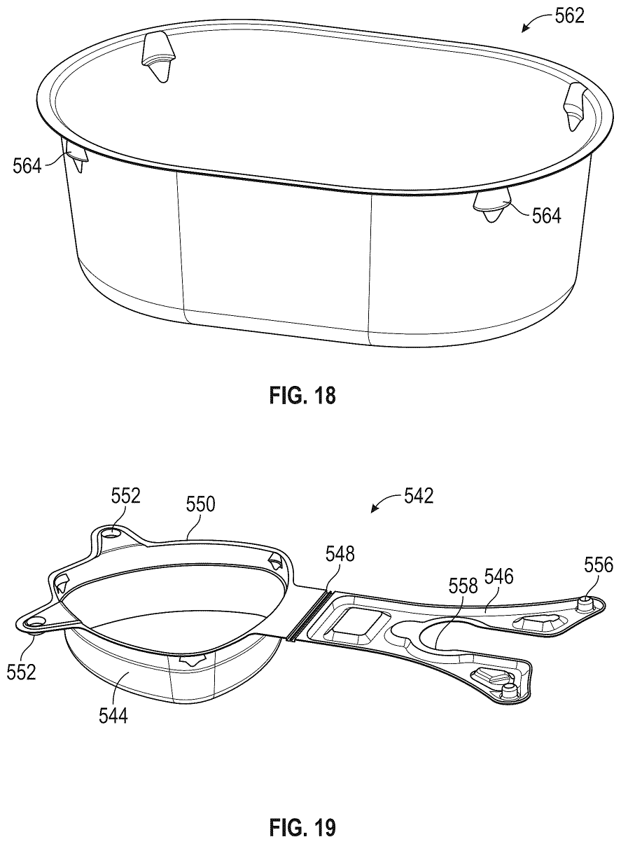

In some examples, the tray can include first and second removable bowls (FIGS. 18 and 19) that are configured to receive a fluid during the preparation procedure.

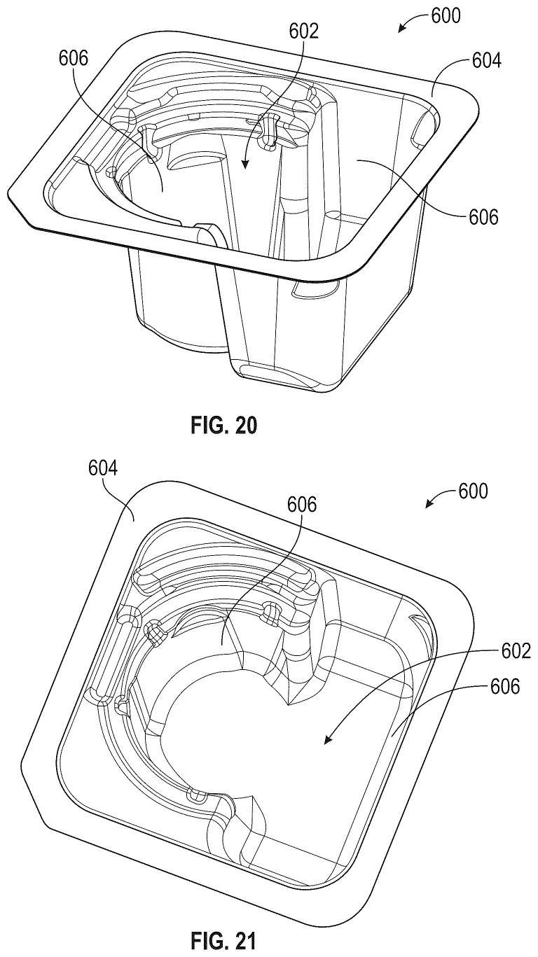

The prosthetic valve can be packaged separately from the delivery apparatus, such as with the package shown in FIGS. 20 and 21.

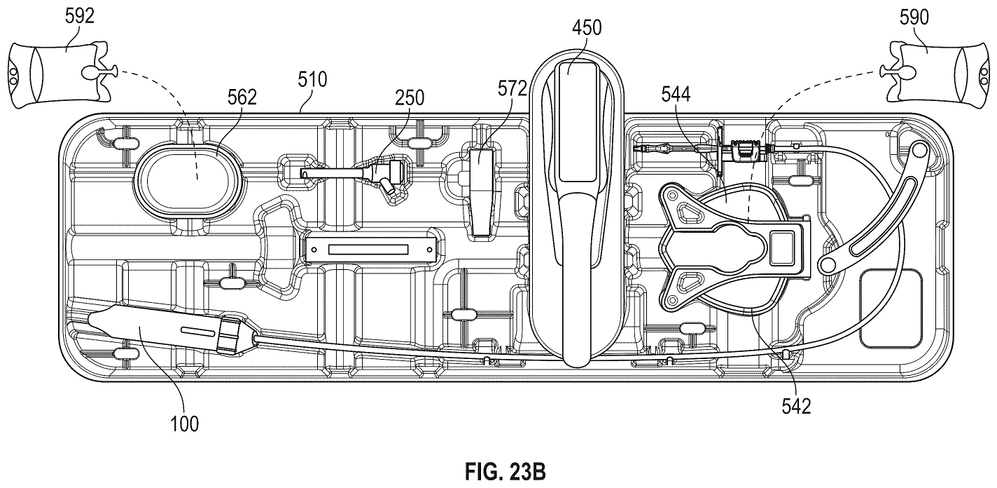

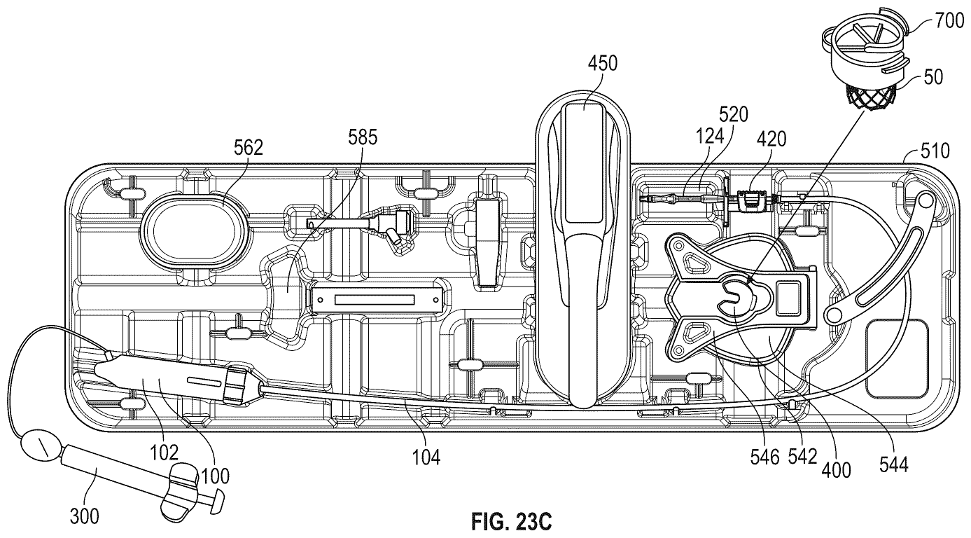

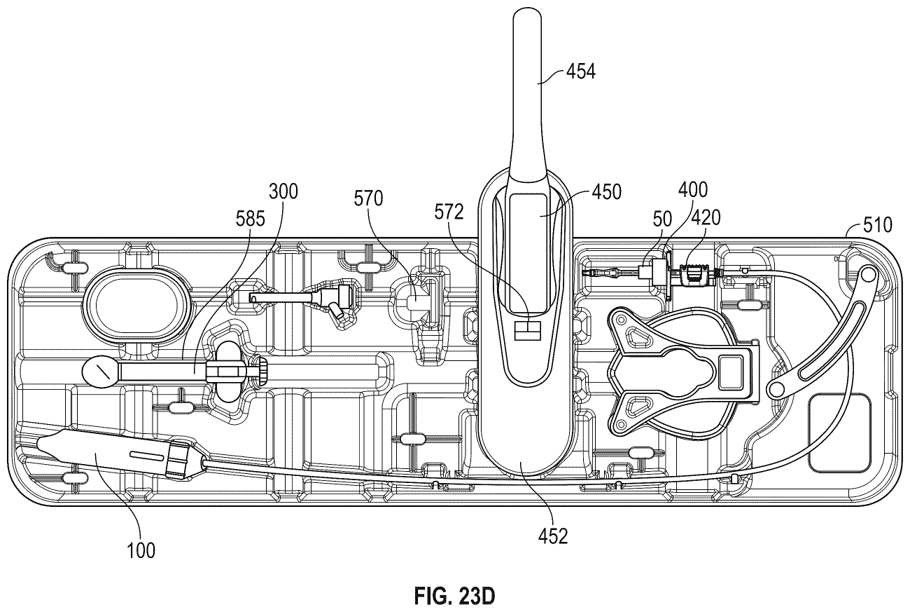

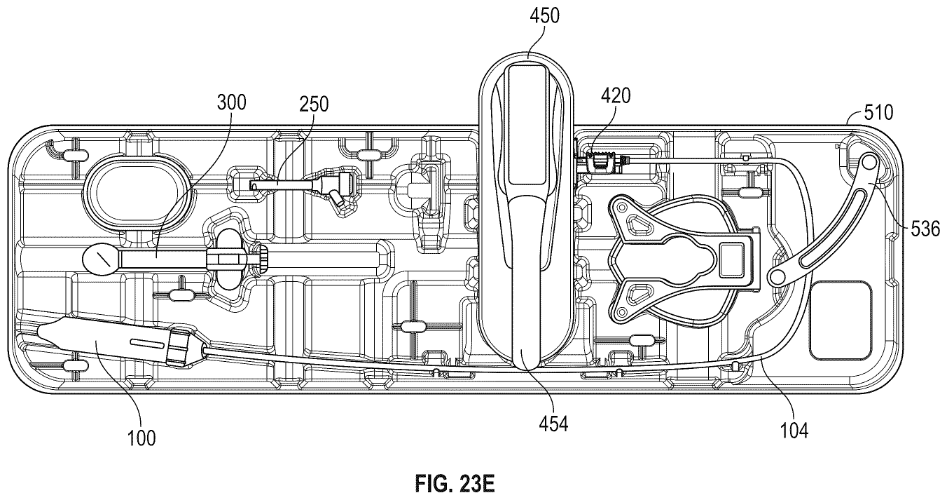

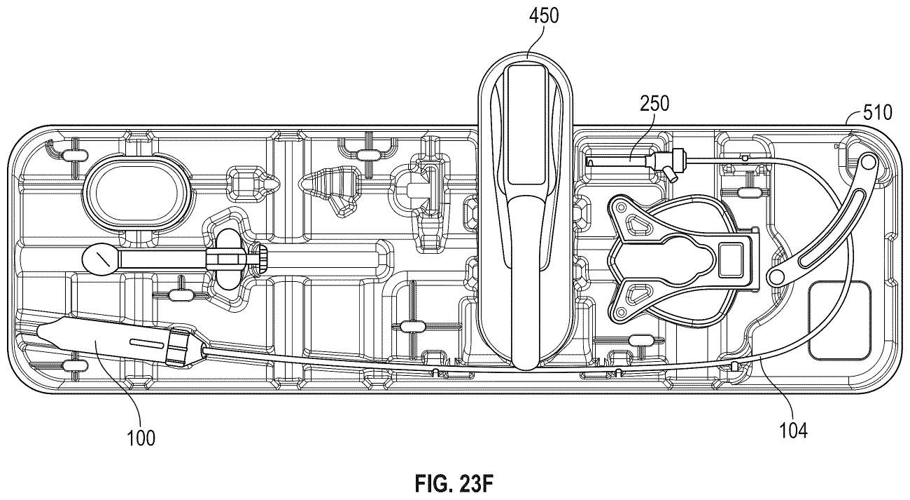

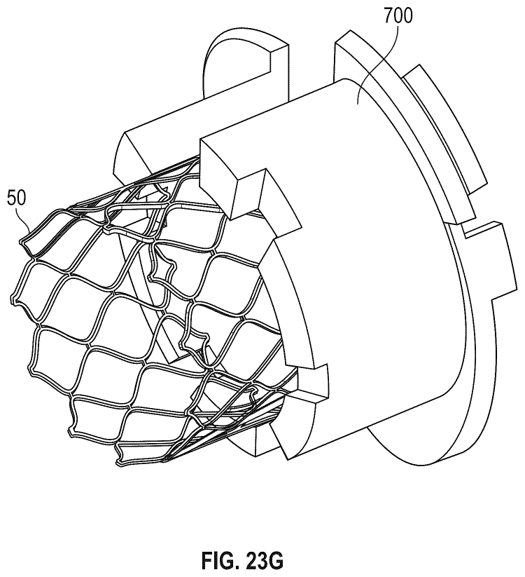

FIGS. 23A-23G show an exemplary procedure for preparing a prosthetic valve and delivery apparatus for implantation into a patient using the tray of FIGS. 11-14.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIGS. 1A and 1B show an exemplary prosthetic valve 50, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO 2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 50 can have three main components: a stent or frame, 52, a valvular structure 54, and a sealing member 56 (FIG. 1A). FIG. 1B is a perspective view of the prosthetic valve 50 with the components on the outside of the frame 52 (including the sealing member 56) shown in transparent lines for purposes of illustration. The prosthetic valve 50 can have an inflow end 66 and an outflow end 68.

The valvular structure 54 can comprise three leaflets 60, collectively forming a leaflet structure, which can be arranged to collapse in a tricuspid arrangement, although in other examples there can be greater or fewer number of leaflets (e.g., one or more leaflets 60). In some examples, the leaflets 60 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

Each leaflet 60 can be coupled to the frame 52 along its inflow edge 62 (the lower edge in the figures; also referred to as “cusp edges”) and at commissures 64 of the valvular structure 54 where adjacent portions (e.g., commissure tabs) of two leaflets are connected to each other. In some examples, the commissures 64 can comprise an attachment member (e.g., comprising fabric, flexible polymer, or the like) arranged across a cell (e.g., commissure cell) of the frame 52, the cell formed by struts of the frame. The attachment member can be secured to the struts of the frame forming the cell and the adjacent portions of the two leaflets can be connected to the attachment member to form the commissure 64 (e.g., as shown in FIGS. 3-8, as described further below).

In some examples, a reinforcing element or connecting skirt, such as a fabric strip, can be connected directly to the cusp edges of the leaflets and to the struts of the frame to couple the cusp edges of the leaflets to the frame.

The frame 52 can be made of any of various suitable plastically-expandable materials (e.g., stainless steel, etc.) or self-expanding materials (e.g., Nitinol). When constructed of a plastically-expandable material, the frame 52 (and thus the prosthetic valve 50) can be crimped to a radially collapsed configuration on a delivery apparatus (e.g., catheter) and then expanded inside a patient by an inflatable balloon or equivalent expansion mechanism. Various crimping devices can be used to crimp the prosthetic valve 50 and the other prosthetic valves described herein around the delivery apparatus, such as the crimping devices described in U.S. Pat. No. 7,530,253, which is incorporated herein by reference.

In some instances, the prosthetic valve 50 can be crimped directly onto the inflatable balloon of the delivery apparatus, such that the prosthetic valve 50 is axially aligned with and disposed radially outward of the balloon during advancing the prosthetic valve on the delivery apparatus to the implantation site, for example as described in PCT Publication No. WO2022/046585, which is incorporated herein by reference. In alternate instances, the prosthetic valve 50 can be crimped onto the delivery apparatus axially offset from the balloon, and then moved over the balloon at the implantation site, prior to inflation of the balloon and radial expansion of the prosthetic valve, such as described in U.S. Pat. No. 9,339,384, which is incorporated herein by reference.

When constructed of a self-expandable material, the frame 52 (and thus the prosthetic valve 50) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Suitable plastically-expandable materials that can be used to form the frames disclosed herein (e.g., the frame 52) include, metal alloys, polymers, or combinations thereof. Example metal alloys can comprise one or more of the following: nickel, cobalt, chromium, molybdenum, titanium, or other biocompatible metal. In some examples, the frame 52 can comprise stainless steel. In some examples, the frame 52 can comprise cobalt-chromium. In some examples, the frame 52 can comprise nickel-cobalt-chromium. In some examples, the frame 52 comprises a nickel-cobalt-chromium-molybdenum alloy, such as MP35N™ (tradename of SPS Technologies), which is equivalent to UNS R 30035 (covered by ASTM F562-02). MP35N™/UNS R30035 comprises 35% nickel, 35% cobalt, 20% chromium, and 10% molybdenum, by weight.

The frame 52 in the illustrated example comprises a plurality of circumferentially extending rows of angled struts 72 defining rows of open cells 74 (or openings) of the frame. The frame 52 can have a cylindrical or substantially cylindrical shape having a constant diameter from the inflow end 66 to the outflow end 68 of the frame 52 as shown, or the frame 52 can vary in diameter along the height of the frame, as disclosed in U.S. Patent Publication No. 2012/0239142, which is incorporated herein by reference.

The frame 52, at each of the inflow end 66 and the outflow end 68, may comprise a plurality of apices 80 spaced apart from one another around a circumference of the frame 52.

The sealing member 56 in the illustrated example is mounted on the outside of the frame 52 and functions to create a seal against the surrounding tissue (e.g., the native leaflets and/or native annulus) to prevent or at least minimize paravalvular leakage. The sealing member 56 can comprise an inner layer 76 (which can be in contact with the outer surface of the frame 52) and an outer layer 78. The sealing member 56 can be connected to the frame 52 using suitable techniques or mechanisms. For example, the sealing member 56 can be sutured to the frame 52 via sutures that can extend around the struts 72 and through the inner layer 76. In alternative examples, the inner layer 76 can be mounted on the inner surface of the frame 52, while the outer layer 78 is on the outside of the frame 52.

The outer layer 78 can be configured or shaped to extend radially outward from the inner layer 76 and the frame 52 when the prosthetic valve 50 is deployed. When the prosthetic valve is fully expanded outside of a patient's body, the outer layer 78 can expand away from the inner layer 76 to create a space between the two layers. Thus, when implanted inside the body, this allows the outer layer 78 to expand into contact with the surrounding tissue.

Additional details regarding the prosthetic valve 50 and its various components are described in U.S. Patent Publication No. 2018/0028310, which is incorporated herein by reference.

FIG. 2 shows a delivery apparatus 100, according to an example, that can be used to implant an expandable prosthetic heart valve (e.g., the prosthetic valve 50 of FIGS. 1A and 1B and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in PCT Publication No. WO2022/046585, which is incorporated by reference herein.

A delivery system for a prosthetic valve, such as the prosthetic valve 50 of FIGS. 1A and 1B, can comprise a plurality of components. For example, in some instances, the delivery system can comprise the prosthetic valve, a delivery apparatus (e.g., the delivery apparatus 100 in FIG. 2), and a crimping device. In some instances, the delivery system can additionally or alternatively comprise a sheath, a loader, and/or a syringe or alternate flushing or de-airing device. In some instances, the delivery system can additionally or alternatively comprise a valve holder and positioning device for crimping the prosthetic valve onto the delivery apparatus in a pre-determined orientation relative to the delivery apparatus. FIGS. 3-9 show examples of a sheath (FIG. 3), a loader (FIG. 4), a flushing device or syringe (FIG. 5), a valve holder (FIG. 6), a positioning device (FIGS. 7 and 8), and a crimping device (FIG. 9) that may be part of a delivery system for a prosthetic valve. It should be noted that these components are exemplary and similar components with alternate configurations or additional components not shown in FIGS. 3-9 (e.g., a crimp stopper, or the like) can be part of the delivery system and used in the preparation procedure described herein.

Turning first to FIG. 3, an exemplary sheath 200 (which can be referred to as an introducer sheath) that is configured to be inserted into a blood vessel of a patient and receive a shaft of a deliver apparatus for a prosthetic device therethrough is shown. As such, the sheath 200 can be used to introduce and at least partially guide a delivery apparatus (such as the delivery apparatus 100 of FIG. 2) into a blood vessel of a patient.

The sheath 200 can comprise a housing 202 (which can also be referred to as a “hub”) and a sleeve 204 (or shaft) extending distally from the housing 202. The housing 202 can house one or more valves. In some examples, the one or more valves may include a sealing valve. A flush port 206, which may include a stopcock in some instances, can extend outward and away from the housing 202.

In some examples, a tapered dilator 220 can extend distally from a distal end of the sleeve 204. In some instances, the dilator 220 can help to pre-dilate the vessel of the patient.

In use, the sleeve 204 (and the dilator 220, when included) is inserted into a body vessel (e.g., the femoral artery) while the housing 202 remains outside the body. In some embodiments, the housing 202 may be referred to as or be part of a proximal portion of the sheath 200.

A delivery apparatus, such as the delivery apparatus 100 of FIG. 2, is inserted through a proximal opening 208 in the housing 202, the one or more valves within the housing 202, the sleeve 204, and into the body vessel. The one or more valves of the housing 202 may sealingly engage the outer surface of an outer shaft of the delivery apparatus (for example, outer shaft 104) to minimize blood loss.

In some applications, a loader device or loader 250 (as shown in FIG. 4) can be placed over the distal end portion of the delivery apparatus and the prosthetic valve radially compressed thereon before the distal end portion of the delivery apparatus is inserted into the housing 202 of the sheath 200. The loader 250 can prevent the one or more valves inside the housing 202 from directly contacting the prosthetic valve mounted on the delivery apparatus as it is pushed through the sheath 200.

As shown in FIG. 4, the loader 250 can comprise a larger diameter hub 252 and a sleeve 254 (or shaft) extending from the hub 252. The sleeve 254 can be configured to receive the distal end portion of the delivery apparatus, including the radially compressed prosthetic heart valve mounted thereon.

Further details regarding exemplary introducer sheaths that can be used in a prosthetic device delivery system are disclosed in U.S. Pat. No. 8,790,387 and U.S. Patent Publication No. 2019/0307589, both of which are incorporated herein by reference.

As introduced above, during a system preparation procedure, prior to inserting the introducer sheath and delivery apparatus into a patient, the delivery apparatus can be de-aired (e.g., via flushing). For example, a syringe, such as the exemplary syringe 300 shown in FIG. 5, can be attached to a flushing port of a delivery apparatus (e.g., the second port 140 of the delivery apparatus 100 of FIG. 2).

As shown in FIG. 5, the syringe 300 includes a barrel 302, plunger 304, and hub 306 (e.g., in the form of a luer lock). The hub 306 is configured to attach to the flushing port of the delivery apparatus.

In some instances, the syringe 300 can further include a finger flange 308, a volume scale 310, and/or fluid window 312. It should be noted that the configuration of syringe 300 is exemplary and various fluid syringes can be used to flush and/or de-air the delivery apparatus.

In some examples, a mounting assembly can be used to mount and crimp a prosthetic valve onto a valve mounting portion of a delivery apparatus, in conjunction with a crimping device, in a predetermined position and/or orientation relative to the delivery apparatus. An exemplary mounting assembly is shown in FIGS. 6-8 and an exemplary crimping device 450 that can be used with one or more components of the mounting assembly is shown in FIG. 9.

The mounting assembly can comprise a valve holder 400 (FIG. 6) that is configured to hold a prosthetic valve and interface with and position the prosthetic valve within a crimping device 450 (FIG. 9). For example, as shown in FIG. 6, the valve holder 400 (which can also be referred to as a support body) can comprise a coupling portion 402 that is configured to be inserted into and/or coupled to a crimping device, such as the crimping device 450 shown in FIG. 9. The valve holder 400 can also comprise a support portion 404 that is configured to hold or support the prosthetic valve (e.g., the prosthetic valve 50 of FIGS. 1A and 1B). It should be noted that only the frame 52 of the prosthetic valve 50 is shown in FIG. 6 for the purpose of illustration only, and in use the entire prosthetic valve 50 can be mounted around the support portion 404 with the leaflets 60 being supported on an exterior of the support portion 404

In some examples, when the valve holder 400 is coupled with the crimping device, the support portion 404 can be positioned between one or more leaflets of the prosthetic valve and the delivery apparatus (e.g., delivery apparatus 100). As such, the support portion can support the leaflets in an open position.

The coupling portion 402 can comprise an alignment member 406 that is configured to rotationally align the valve holder 400 with the crimping device 450 in a predetermined orientation. In some instances, the alignment member 406 can comprise an axially extending protrusion that extends axially outward from a first end of the coupling portion 402 toward the support portion 404.

In some examples, the alignment member can have different configurations, such as a recess or alternate alignment feature that is configured to mate with a corresponding mating interface of the crimping device 450 (e.g., a cut-out portion on a second side of the crimping device).

The support portion 404 can have a tapered shape that tapers radially inward in a direction away from the coupling portion 402. For example, a diameter of the support portion 404 can decrease as it extends away from the coupling portion 402. In some instances, the support portion 404 can have a conical shape, as shown in FIG. 6.

The tapered shape of the support portion 404 can allow the valve holder 400 to slide axially away from the prosthetic valve (and the crimping device) during and as a result of the crimping device 450 crimping the prosthetic valve.

As shown in FIG. 6, the valve holder 400 can include a central aperture 408 defining a central channel. The central aperture 408 may be configured for the delivery apparatus to extend therethrough.

In use, the prosthetic valve (frame 52 shown in FIG. 6) can be slid distally onto the support portion 404 of the valve holder 400. In some examples, to align the prosthetic valve in a desired circumferential orientation around the support portion 404, and to space the prosthetic valve from the coupling portion 402 at a desired spacing, a ring body 410 (which can also be referred to as an alignment ring) can be utilized and positioned upon the valve holder 400 (FIG. 6).

The ring body 410 can be configured to couple to and extend around the valve holder 400 (FIG. 6). In some instances, an alignment guide can be positioned on the ring body 410. The alignment guide can comprise one or more indicators 412 (which may also be referred to as alignment markers) configured to indicate a desired circumferential (e.g., rotational) position of selected elements (e.g., commissures) of the prosthetic valve relative to the ring body 410 and the valve holder 400 (FIG. 6). By aligning the selected elements (e.g., commissures) of the prosthetic valve with the indicators 412 of the ring body 410, the selected elements can be positioned in a desired circumferential orientation relative to the ring body 410 and thus relative to the valve holder 400 (e.g., relative to the alignment member 406).

The ring body 410 is removed prior to coupling the valve holder 400 with the crimping device 450. The alignment member 406 can then rotationally align the valve holder 400 with the crimping device 450, and thus place the selected elements of the prosthetic valve in a desired rotational orientation within the crimping device 450. As a result, the prosthetic valve can be crimped onto the delivery apparatus at a predetermined circumferential orientation relative to the delivery apparatus.

In some examples, the mounting assembly can further comprise a positioning device 420 (FIG. 7) configured to couple to a portion (e.g., distal end portion) of the delivery apparatus 100 (or an alternate delivery apparatus), proximal to the valve mounting portion 124.

The positioning device 420 includes a body 422 including a first portion 424 and a second portion 426 pivotably coupled to one another via a hinge 428. The body 422 can include a central channel 430 (FIG. 6) that is configured to receive the intermediate shaft 106 (or another shaft portion, such as the outer shaft 104) of the delivery apparatus 100 (FIG. 8).

The first portion 424 of the body 422 can include a flange portion 432 extending radially outward therefrom and arranged at a distal end of the positioning device 420. The flange portion 432 can include one or more mating elements that are configured to mate with correspondingly shaped mating features in a side surface (e.g., proximal face) of a crimping device (e.g., crimping device 450). In some examples, as shown in FIG. 7, the mating elements are configured as circumferentially extending extension portions 434.

As shown in FIG. 8, the positioning device 420 is clamped around the intermediate shaft 106 at a location proximal to and adjacent to a proximal end portion of the balloon 118.

FIG. 9 shows an exemplary crimping device 450 that can be used with the mounting assembly to crimp (radially compress) the prosthetic valve around a distal end portion of the delivery apparatus 100. The crimping device 450 can include a base 452, an actuator in the form of a handle 454, and a channel 456 for the prosthetic valve and the delivery apparatus to be inserted into.

The crimping device 450 can include mating interfaces, on opposite sides of the crimping device 450, that are configured to receive and mate with corresponding mating interfaces on the valve holder 400 and the positioning device 420 of the mounting assembly. FIG. 9 shows a view of a first side 458 of the crimping device 450 that is configured to interface with the positioning device 420.

For example, the first side 458 can include mating structures 460 (e.g., cut-outs) that can be configured to mate with the positioning device 420 (FIG. 9). In some instances, the mating structures 460 can be configured to receive and mate with the extension portions 434 of the positioning device 420.

An opposite, second side of the crimping device 450 (behind the first side 458 in the view of FIG. 9) can include one or more additional mating structures that can be configured to receive and mate with the coupling portion 402 of the valve holder 400 (e.g., the alignment member 406).

The crimping device 450 can further include a rotatable body 462 configured to be rotated with rotation of the handle 454 about axis 453. The crimping device 450 may operate by a plurality of pressing surfaces 464 surrounding the channel 456 and being configured to apply a compressive force to radially compress a prosthetic valve positioned within the channel 456 (e.g., prosthetic valve 50 of FIGS. 1A and 1B). The pressing surfaces 464 may be configured such that as the rotatable body 462 is rotated, a body presses and moves the pressing surfaces 464 towards the center of the channel 456 and the diameter of the channel 456 reduces, thereby compressing the prosthetic heart valve arranged within the channel 456.

The configuration of a crimping device can be varied in alternate examples.

Additional details on the crimping device and the mounting assembly (e.g., including the valve holder and the positioning device) shown in FIGS. 6-9, and additional examples of such components, can be found in PCT Publication No. WO2022/046585, as already incorporated by reference above.

The components of a delivery system for a prosthetic valve, which may include all or a portion of the components shown in FIGS. 3-9 and/or additional or differently configured components than those described above (such as the valve holder 700 shown in FIG. 23G) can be packaged in multiple packages for shipping, storage, and/or transport and then prepared and assembled together on a table of an operating room, prior to a valve implantation procedure.

For example, the delivery apparatus (e.g., delivery apparatus 100 of FIG. 2) can be packaged in a first package 500 (or packaging or packaging assembly), as shown in FIG. 10. The first package 500 can comprise an outer shipping packaging 502 (or box), a tray 510 (or packaging tray) configured to receive and retain the delivery apparatus therein, and a retainer 508 configured to fit or nest over the tray 510, thereby retaining the contents of the tray 510 within the cavities of the tray 510.

In some instances, the first package 500 can further comprise a pouch or sleeve 506 configured to hold the assembled together tray 510 and retainer 508 therein. In some examples, the sleeve 506 can comprise a polymeric material such as nylon, polyethylene, and/or the like.

In some instances, the first package 500 can further comprise an inner box or packaging 504 that is configured to receive the tray 510 and retainer 508 assembled together in the sleeve 506 and that is configured to be received within the outer shipping packaging 502.

In some instances, a positioning device (e.g., positioning device 420 of FIG. 7) can be coupled on a distal end portion of the delivery apparatus for shipping, and thus arranged within the tray 510 (as described further below).

In some instances, a loader (e.g., the loader 250 of FIG. 4) can also be packaged within the first package 500 (for example, within the tray 510).

In some instances, additional components can be packaged within the tray 510 of the first package 500, such as a crimp stopper and one or more fluid bowls, as described further below.

In some examples, one or more components inside the first package 500 can be sterilized before packaging and shipping. For example, in some instances, the tray 510 and retainer 508 are sterilized and sealed within the sleeve 506 which is also sterile.

Additional components of the delivery system can be packaged separately in additional packages for shipping and transport to an implantation facility. For example, in some instances, a sheath (e.g., sheath 200 of FIG. 3), a syringe (e.g., syringe 300 of FIG. 5), a crimper (e.g., crimping device 450 of FIG. 9), and a prosthetic valve (e.g., prosthetic valve 50 of FIGS. 1A and 1B) and valve holder (e.g., valve holder 400 of FIG. 6 or valve holder 700 of FIG. 23G) can be packaged in additional packages.

In some examples, the first package 500 and the additional packages can all be packaged together in one larger box or container for shipping and transport together to an implantation facility.

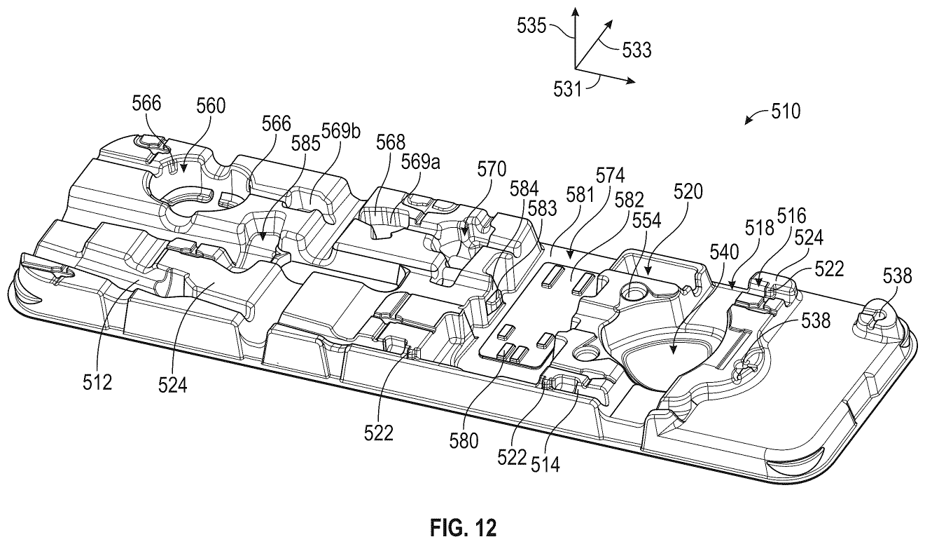

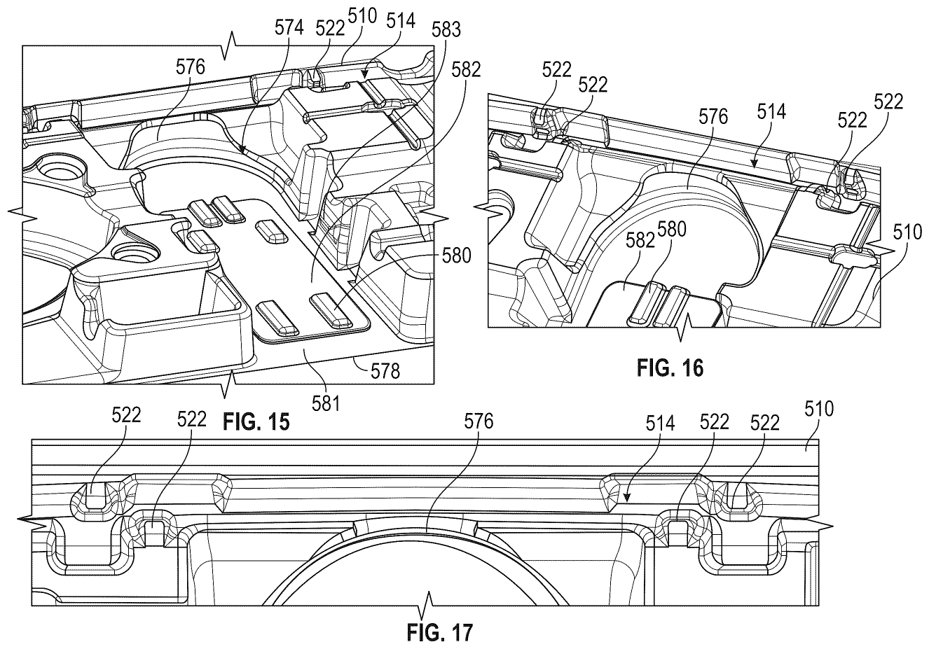

FIGS. 11-22 illustrate the tray 510 in more detail, as well as components included in the tray 510 or couplable to the tray 510. For example, FIGS. 11 and 12 show top and perspective views, respectively, of the tray 510 alone (without additional components arranged therein) and FIGS. 13 and 14 show top and perspective views, respectively, of the tray 510 with a delivery apparatus and a crimping device disposed within the tray 510. FIGS. 15-17 show various detail views of portions of the tray 510 configured to interface with the crimping device and FIGS. 18-19 show various bowls or vessels configured to be received within specified portions of the tray 510. Additionally, FIGS. 20 and 21 show an exemplary packaging for a prosthetic valve and FIG. 22 shows the retainer 508 in further detail.

The tray 510 has a length 521 and can be sectioned into a first end portion 523 extending along a third of the length 521, a second end portion 525, opposite the first end portion 523, extending along a third of the length 521, and a middle portion 527 extending along a third of the length 521 from the first end portion 523 to the second end portion 525 (FIG. 13).

As shown in FIGS. 11 and 12, the tray 510 comprises a plurality of spaces, cavities, or recesses that are configured to receive one or more components of the delivery system (e.g., the delivery apparatus and crimping device) and/or additional components couplable with the tray 510 (e.g., removable fluid bowls). The tray 510 can also comprise one or more retaining features (e.g., in the form of tabs, snaps, straps, or alternate locking features) that are configured to hold the one or more components of the delivery system within their corresponding recesses.

The tray 510 can comprise a first set of recesses that are configured to receive components of the delivery system to be shipped inside the tray 510.

For example, as shown in FIGS. 11 and 12, the tray 510 can comprise a set of recesses configured to receive a delivery apparatus (e.g., delivery apparatus 100, as shown in FIGS. 13 and 14). The set of recesses can include a first recess 512 configured to receive a handle (e.g., handle 102) of the delivery apparatus and a second recess 514 configured to receive a proximal portion of a shaft of the delivery apparatus (e.g., shaft 104). The second recess 514 can comprise one or more connected recesses that hold and retain the proximal portion of the shaft of the delivery apparatus therein with one or more retaining features 522. In some instances, the retaining features 522 can comprise single tabs and/or pairs of opposing tabs that extend over a portion of the second recess 514, each pair of opposing tabs extending from a different side of the second recess 514 (toward one another but spaced apart in an axial direction of the delivery apparatus shaft). As such, the proximal portion of the delivery apparatus shaft can “snap” into the second recess 514 and be held or retained therein by the retaining features 522. Detail views of pairs of retaining features 522 disposed proximate to a tenth recess 574 (as described further below) are shown in FIGS. 16 and 17.

The set of recesses configured to receive the delivery apparatus can also include a third recess 516 (FIGS. 11 and 12) configured to receive a distal end portion of the delivery apparatus (e.g., a shaft portion that is proximal to the positioning device 420, as shown in FIGS. 13 and 14). The third recess 516 can include one more retaining features 522 (FIGS. 11 and 12). In some instances, the retaining features 522 of the third recess 516 can comprise a plurality of tabs spaced apart from one another in an axial direction of the delivery apparatus shaft.

The tray 510 can comprise a fourth recess 518 configured to receive a positioning device coupled to a distal end portion of the delivery apparatus (e.g., the positioning device 420 coupled to the distal end portion of the delivery apparatus 100, as shown in FIGS. 13 and 14). In some instances, the fourth recess 518 can be depressed further (deeper) into a raised surface 524 of the tray 510 that the third recess 516 (see FIG. 12).

The tray 510 can comprises a fifth recess 520 configured to receive a distal end portion of the delivery apparatus that includes a valve mounting portion (e.g., the valve mounting portion 124 of the distal end portion of the delivery apparatus, as shown in FIGS. 13 and 14).

As shown in FIGS. 13 and 14, the arrangement of the recesses described above for the delivery apparatus results in a curved section 526 of the shaft 104 of the delivery apparatus that forms a bend. For example, the curved section 526 forming the bend in the shaft 104 is disposed closer to the distal end (or valve mounting portion 124) of the delivery apparatus than the handle 102 of the delivery apparatus.

In some examples, the curved section 526 of the delivery apparatus shaft forms a 180 degree bend along a distal half of the shaft 104. Additionally, as shown in FIGS. 13 and 14, a proximal half of the shaft 104 can be substantially straight.

As a result, the distal end portion of the delivery apparatus including the valve mounting portion 124 is positioned on an opposite side of the tray 510 than a proximal end portion of the delivery apparatus which includes the handle 102.

For example, the first recess 512 and second recess 514 are disposed on a first side 528 of the tray 510 while the third recess 516, fourth recess 518, and fifth recess 520 are disposed on an opposite, second side 530 of the tray 510. As such, the curved section 526 of the shaft is positioned in the first end portion 523 of the tray 510 and extends along and adjacent to a third side 532 of the tray 510 (the third side 532 disposed opposite a fourth side 534 of the tray 510 and the third and fourth sides 532 and 534 each extending between the first and second sides 528 and 530) (FIGS. 13 and 14). The third side 532 and the fourth side 534 of the tray 510 can be referred to as opposite first and second ends of the tray 510.

In some examples, the tray 510 comprises a strap 536 (FIGS. 13 and 14) that couples to coupling features 538 on the raised surface 524 of the tray 510 (FIGS. 11 and 12). In some examples, as shown in FIGS. 11 and 12, the coupling features 538 are depressed into the raised surface 524 and the strap 536 can comprise complementary mating features at its ends that snap into, or mate in another way to, the coupling features 538. As a result, the distal end portion of the shaft 104 of the delivery apparatus can be retained against (and/or within) the tray 510 (FIGS. 13 and 14). This can be beneficial during a prep procedure (e.g., during flushing the delivery apparatus and/or crimping the valve onto the delivery apparatus) so that the delivery apparatus remains within the tray 510 (and thus remains within a sterile environment).

In some instances, the strap 536 is curved and curves from a corner of the tray 510 that extends between the second side 530 and the third side 532 of the tray 510 toward the middle portion 527 of the tray 527.

In some examples, the tray 510 can comprise a sixth recess 540 (FIGS. 11 and 12) configured to receive a removable first bowl 542 (FIGS. 13, 14, and 19). FIG. 19 shows the first bowl 542 separate from the tray 510 and in an unfolded configuration. The first bowl 542 includes a bowl portion 544 and a holder portion 546. The holder portion 546 is foldable relative to the bowl portion 544 (and vice versa) via a crease or folding line 548. The folding line 548 can be a thinner material or indented such that the holder portion 546 can fold over an open top 550 of the bowl portion 544.

The bowl portion 544 can be shaped such that it fits within the sixth recess 540. In some examples, the bowl portion 544 has a triangular shape. In some examples, the bowl portion 544 (and the sixth recess 540) can have a different shape such as rectangular, square, circular, or the like.

In some examples, the bowl portion 544 can comprise one or more coupling features 552 that are configured to couple to or mate with complementary coupling features 554 (e.g., depressions) disposed in the tray 510 adjacent to the sixth recess 540 (FIGS. 11 and 12). As a result, the bowl portion 544 can be mated with and held within (e.g., by press fit, snap fit, or other coupling means) the sixth recess 540 by the coupling features 552 and complementary coupling features (FIGS. 13 and 14).

In some examples, the holder portion 546 can include coupling features 556 (e.g., in the form of nubs or protrusions) which fit within and mate with an opposite side of the coupling features 552 of the bowl portion 544 when the holder portion 546 is folded over the bowl portion 544 (as shown in FIGS. 13 and 14).

In some instances, the first bowl 542 is a saline bowl configured to receive a saline solution therein during a prep procedure. A prosthetic valve mounted on a valve holder (e.g., valve holder 700 of FIG. 23G or valve holder 400 of FIG. 6) can be inserted into the bowl portion 544 through an opening 558 in the holder portion 546 and an end of the valve holder can be held above/by the opening 558 such that the prosthetic valve is suspended within the bowl portion 544 (as shown in FIG. 14) and can be soaked prior to mounting onto the delivery apparatus.

In some examples, the tray 510 can comprise a seventh recess 560 (FIGS. 11 and 12) configured to receive a removable second bowl 562 (FIGS. 13, 14, and 18). FIG. 18 shows the second bowl 562 separate from the tray 510. The second bowl 562 is shaped to fit within the seventh recess 560.

In some instances, the second bowl 562 can comprise one or more mating features 564 (or coupling features) that are configured to mate with (e.g., snap and/or fit into) complementary mating features 566 on the seventh recess 560 (FIG. 12). For example, the mating features 564 can be protrusions on an outer surface of the second bowl 562 that are configured to fit into and mate with the complementary mating features 566 which can be in the form of depressions in a sidewall of the seventh recess.

In some instances, the second bowl 562 is a contrast bowl configured to receive a contrast solution during the prosthetic valve and delivery apparatus prep procedure.

Both the first bowl 542 and the second bowl 562 are removable from their respective recesses in the tray 510 in order to be filled with and/or emptied of fluid. By incorporating removable fluid bowls with the tray 510, the tray 510 does not need to be moved to fill or empty cavities with fluid, thereby leaving the tray 510 undisturbed and simplifying the prep procedure.

In some examples, instead of being removable, the fluid bowls 542 and 562 can be integrated (formed as one piece) with the tray 510.

In some examples, the tray 510 can comprise an eighth recess 568 (FIGS. 11 and 12) configured (shaped) to receive a loader (e.g., the loader 250 shown in FIG. 4) (FIGS. 13 and 14). As shown in FIGS. 11-14, in some instances, the eight recess 568 can comprise two recess portions separated from one another where a first recess portion 569a is configured to receive a first end (e.g., the hub 252) of the loader 250 and a second recess portion 569b is configured to receive a second end (e.g., the sleeve 254) of the loader 250.

In some examples, the tray 510 can comprise a ninth recess 570 (FIGS. 11 and 12) for a crimp stopper 572 (FIGS. 13 and 14). For example, the crimp stopper 572 can be packaged with the tray 510 and then used with (e.g., attached to) the crimping device during the prosthetic valve and delivery apparatus prep procedure to crimp the prosthetic valve onto the delivery apparatus (e.g., as shown in FIG. 23D, described further below). By including the crimp stopper 572 in the tray 510 and the first package 500 in some examples, the crimp stopper 572 can be more quickly located and used during the prep procedure.

In some examples, the ninth recess 570 can be shaped to receive a differently shaped crimp stopper or a different component of the delivery system. In some examples, the tray 510 may not include the ninth recess 570 (for example, if the delivery system does not include a crimp stopper or the crimp stopper is packaged separately).

The tray 510 can also comprise a second set of recesses that are configured to receive components of the delivery system that are shipped, stored, or otherwise transported separately from the tray 510 but are used during a preparation procedure for preparing the delivery apparatus and prosthetic valve for an implantation procedure (referred to herein as the “prep procedure” or “prep procedure for the prosthetic valve and delivery apparatus”).

For example, the tray 510 can comprise a tenth recess 574 (FIGS. 11 and 12) that is configured to receive a crimping device (e.g., the crimping device 450 shown in FIG. 9) (FIGS. 13 and 14). The tenth recess 574 can be depressed into the tray from the raised surface 524 and have a width (in a direction parallel to the first and second sides 528 and 530) and length (in a direction parallel to the third and fourth sides 532 and 534) that is sized to receive the base 452 of the crimping device 450 (or a base of another crimping device).

In some instances, the tenth recess 574 can have a closed end 576 (which may be rounded to match a shape of the base 452, as shown in FIG. 11) and an open end 578 such that an end of the crimping device 450 can extend out the open end 578 and past the second side 530 of the tray 510 (as shown in FIGS. 13 and 14).

FIGS. 15-16 show detail views of portions of the tray 510 including the tenth recess 574. As shown in FIGS. 11-12 and 15-16, the tenth recess 574 can include one or more or a plurality of protrusions 580 that extend away from a base 581 of the tenth recess 574 (toward the raised surface 524) that are configured to mate with the base 452 of the crimping device 450 and position the crimping device 450 with a specified horizontal and longitudinal orientation within the tenth recess 574 (relative to a longitudinal direction 531 (or lateral direction), horizontal direction 533, and vertical direction 535, as shown in FIG. 12).

In some instances, the protrusions 580 can be a plurality of spaced apart protrusions 580 that are grouped into one or more groups along the tenth recess 574.

In some examples, the protrusions 580 protrude outward from a raised surface 582 extending from the base 581 of the tenth recess 574. For example, as shown in FIG. 12, the raised surface 582 can be shaped (e.g., rectangular or another complementary shape to the crimping device 450) to receive the base 452 of the crimping device 450 thereon and the protrusions 580 can protrude further outward from the raised surface 582 to further align the crimping device 450 within the tenth recess 574.

As shown in FIGS. 11-12, 15, and 16, in some instances, the protrusions 580 can be rectangular in shape with curved edges. In some instances, the protrusions 580 can have a different shape, such as square, oval, circular, or the like.

In some examples, the tenth recess 574 can also include protrusions or retaining features 583 that protrude outward from a sidewall 584 of the tray 510 that defines the tenth recess 574 (FIGS. 11, 12, and 15). The retaining features 583 can be configured to hold or retain the crimping device 450 within the tenth recess 574 during use (e.g., during crimping), thereby preventing the crimping device 450 from rotating and/or being dislodged from the tenth recess 574 (for example, even as a handle of the crimping device is actuated).

In this way, the retaining features 583 and the protrusions 580 can help to maintain or “lock” the crimping device 450 into the tray 510 such that it does not slide around during the prep procedure (including crimping).

As shown in FIGS. 11-14, the tenth recess 574 can be formed in the middle portion 527 of the tray 510, thereby positioning the crimping device 450 along the middle portion 527 of the tray 510 when the crimping device is coupled with the tray 510 (as shown in FIGS. 13 and 14). The tenth recess 574 and the first, second, third, fourth, and fifth recesses 512, 514, 516, 518, and 520 can be further positioned within the tray 510 such that a valve mounting portion 124 of the shaft of the delivery apparatus 100 is adjacent to the crimping device 450.

In some examples, these recesses are positioned relative to one another within the tray 510 such that the valve mounting portion 124 is adjacent a valve-receiving portion (e.g., channel 456) of the crimping device 450.

In some instances, the recesses are positioned relative to one another within the tray 510 such that a longitudinal axis of the valve mounting portion 124 is parallel to a longitudinal axis of the valve-receiving portion of the crimping device 450 (which can both be parallel to the second side 530 of the tray 510).

In some instances, the axes of the valve mounting portion 124 and the valve-receiving portion of the crimping device 450 can be substantially aligned with one another relative to the second side 530 (longitudinal edge) of the tray 510.

As a result, the distal end portion of the delivery apparatus (the valve mounting portion 124) can be inserted into the channel 456 of the crimping device 450 by sliding the distal end portion of the delivery apparatus a short distance (right to left in FIG. 13) into the channel 456 of the crimping device 450 without having to remove the delivery apparatus from the tray 510. For example, the shaft of the delivery apparatus can remain engaged with the retaining features 522 inside the recesses and just slide axially within the recesses (along an axis of the delivery apparatus) so that the valve mounting portion can reach the channel 456 of the crimping device 450.

In some instances, the strap 536 described above can hold the shaft of the delivery apparatus in place such that the shaft can slide during crimping of the prosthetic valve onto the distal end portion of the delivery apparatus but not move off/outside of the tray 510.

Additionally, in some examples, the first recess 512 can be longer than the handle 102 of the delivery apparatus 100 (see FIG. 13 for example). This can allow the handle 102 to slide axially within the recess 512 as the distal end portion of the delivery apparatus 100 is moved into and out of the crimping device 450.

In some examples, the tray 510 can comprise an eleventh recess 585 (FIGS. 11-14) configured to receive a syringe (e.g., the syringe 300 shown in FIG. 5). For example, the eleventh recess 585 can be shaped to receive the syringe during the prep procedure (as explained further below with reference to FIGS. 23A-23F).

As shown in FIGS. 11-14, the eleventh recess 585 can be disposed adjacent to the fourth side 534 of the tray 510, between the first recess 512 and the seventh recess 560. In some instances, the eleventh recess 585 extends from the fourth side 534 of the tray 510 to the middle portion 527 of the tray 510 that is proximate to the tenth recess 574.

In some examples, the eighth recess 568 can be disposed between the seventh recess 560 and the ninth recess 570 and/or between the seventh recess 560 and the tenth recess 574.

In some examples, the sixth recess 540 can be disposed adjacent to the tenth recess 574, on a side of the tenth recess 574 that is closer to the third side 532 than the fourth side 534 of the tray 510.

In some examples, the sixth recess 540 can be connected to the fourth recess 518.

In some examples, the handle 102 and second bowl are positioned within the second end portion 523 of the tray 510.

In some examples, the tray 510, the delivery apparatus (e.g., the delivery apparatus 100), and the crimping device (e.g., the crimping device 450) can form an assembly (FIGS. 13 and 14).

In some examples, the assembly can further comprise a prosthetic valve.

In some examples, the assembly can further comprise a valve holder for the prosthetic valve.

In some examples, the assembly can further comprise one or more fluid bowls (e.g., bowls 542 and 562) that are removably coupled to the tray 510.

In some examples, the assembly can further comprise one or more of a loader, crimp stopper, and syringe.

The assembly can be used for preparation of a prosthetic valve and the delivery apparatus for a prosthetic valve implantation procedure, as described below with reference to FIGS. 23A-23F.

As introduced above, a prosthetic valve (e.g., prosthetic valve 50), which can be mounted on a valve holder in some instances, can be packaged separately from the tray 510 and then received within the holder portion 546 of the first bowl 542 inside the tray 510 during the prep procedure. FIGS. 20 and 21 show an exemplary packaging 600 (or shipping tray) for containing the prosthetic valve during shipping and storage.

The packaging 600 can include a cavity 602 depressed into the packaging 600 from a top surface 604 of the packaging 600. The cavity 602 can be defined by interior walls 606 of the packaging 600 which are shaped to fit the prosthetic valve. In some examples, the interior walls 606 can be shaped to receive the prosthetic valve mounted on a valve holder (e.g., valve holder 700 of FIG. 23G).

FIG. 22 is a bottom perspective view of the retainer 508 of the package 500 which is shaped to fit over the tray 510. The retainer 508 comprises an exterior surface 507 (FIG. 10), an interior surface 505 (FIG. 22), and a lip 513 (or sidewall) extending from away from the exterior surface 507 around a perimeter of the retainer 508. The lip 513 is shaped to fit over the first, second, third, and fourth sides 528, 530, 532, and 534 of the tray 510.

The interior surface 505 of the retainer 508 comprises depressions 509 that are shaped to fit over components arranged within the tray 510 (e.g., the handle 102 of the delivery apparatus) and protrusions 511 that are shaped to fit into vacant recesses in the tray 510 or over components disposed within recesses of the tray 510 (e.g., components such as the loader that are disposed lower than the raised surface 524 of the tray 510).

Turning now to FIGS. 23A-23F an exemplary method for using the tray 510 as an integrated prep tray for a prosthetic valve and delivery apparatus preparation procedure (“prep procedure”) is shown. For example, the prep procedure may occur at an implantation facility, after shipping the delivery system components to the implantation facility (e.g., the first package 500 and one more additional packages containing components not shipped within the tray 510, such as a crimping device, syringe, and prosthetic valve) and prior to a prosthetic valve implantation procedure where a prosthetic valve mounted on the distal end portion of delivery apparatus is inserted in a vessel of a patient. The implantation facility may include an operating room or procedure room at a hospital or other medical facility.

To begin the prep procedure for preparing the delivery apparatus and prosthetic valve for implantation into a patient, the tray 510 can be removed from the first package 500 and placed on a surface in an operating or procedure room (e.g., a table) (FIG. 23A). In some examples, the tray 510 and its contents, as well as the table or other surface on which the tray 510 is placed may be sterile. For example, the tray 510 and its contents may be sterilized and arranged within sterile packaging (components of the package 500) prior to shipping and storage.

As shown in FIG. 23A, upon opening the package 500 and placing the tray 510 on the surface, the tray 510 can contain a delivery apparatus (e.g., delivery apparatus 100). In some examples, the tray 510 can also contain a loader (e.g., loader 250) and/or a crimp stopper (e.g., crimp stopper 572). The tray 510 can also contain the first bowl 542 in the sixth recess 540 (with the holder portion 546 folded over the bowl portion 544) and the second bowl 562 within the seventh recess 560.

These components are arranged within their respective recesses in the tray 510 as described above. For example, the delivery apparatus is retained within recesses 512, 514, 516, 518, and 518 and the strap 536 is holding the curved portion of the shaft 104 against the tray 510.

A crimping device (e.g., crimping device 450) can then be removed from its packaging and placed in the tenth recess 574 (FIG. 23B). As described above, the crimping device 450 can be held or locked within the tenth recess 574 such that is does not rotate, twist, or otherwise become uncoupled from the tenth recess 574 of the tray 510 during the prep procedure (e.g., during crimping, as shown in FIG. 23E).

After attaching the crimping device 450 to the tray 510, the first bowl 542 and the second bowl 562 can be filled with fluid. For example, in some examples, the bowl portion 544 of the first bowl 542 is filled with a saline solution 590 (e.g., heparinized saline) and the second bowl 562 is filled with contrast 592.

After filling the fluid bowls within the tray 510, the prosthetic valve (e.g., prosthetic valve 50) is positioned within the first bowl 542 to soak in the saline solution (FIG. 23C) (e.g., to rehydrate the prosthetic valve). In some examples, the prosthetic valve is mounted on a valve holder (e.g., valve holder 700 which is shown in greater detail in FIG. 23G) and the prosthetic valve mounted on the valve holder is placed in the first bowl 542 such that the valve is suspended within the bowl portion 544 by the valve holder that is held by the holder portion 546 of the first bowl 542 (FIG. 23C). For example, the holder portion 546 can be shaped to receive the valve holder 700, which is shown in more detail in FIG. 23G.

As also shown in FIG. 23C, the delivery apparatus can be de-aired by flushing fluid from a syringe (e.g., syringe 300). For example, the syringe 300 (filled with fluid) can be attached to a flushing port of the delivery apparatus 100 (such as a flushing port in the handle 102 as shown in FIG. 2 or a flushing port attached to another portion of the delivery apparatus 100) and used to de-air the delivery apparatus.

In some instances, one or more of the recesses of the tray 510 (such as the fifth recess 520) can collect fluid that exits the distal end of the delivery apparatus 100 during the flushing.

After de-airing the delivery apparatus is complete, the syringe 300 can be placed within the eleventh recess 585 of the tray 510 (FIG. 23D). The valve holder 700 (or valve holder 400 in other examples) and valve 50 can then be positioned over the distal end portion of the delivery apparatus 100, adjacent to the positioning device 420 (e.g., such that the valve is over at least a portion of the valve mounting portion 124) (FIG. 23D). In some examples, the valve holder 700 can be locked in place with the positioning device 420.

As shown in FIG. 23D, the handle of the crimping device 450 can be rotated, thereby opening the crimping jaws or surfaces inside the channel of the crimping device 450 (such that it is ready to receive the delivery apparatus and prosthetic valve therein).

In some instances, the crimp stopper 572 can be removed from the ninth recess 570 and attached to the crimping device 450 (FIG. 23D).

Moving to FIG. 23E, the prosthetic valve and valve holder mounted around the delivery apparatus 100 are moved inside the channel of the crimping device 450. As a result, the shaft 104 of the delivery apparatus 100 can slide along the tray 510 but still remain within the tray 510 (e.g., restrained by the strap 536). In some instances, the valve holder and positioning device 420 can be coupled with the mating features on the crimping device 450 (as described above).

The prosthetic valve can then be crimped onto the distal end portion of the delivery apparatus 100 by actuating (e.g., rotating) the handle 454 of the crimping device 450.

After crimping, the distal end portion of the delivery apparatus 100 with the prosthetic valve mounted (crimped) thereon can be removed from the crimping device 450 and then inserted into the loader 250 (FIG. 23F).

In some examples, a stylet inside the delivery apparatus can be removed from the delivery apparatus and the loader 250 can be flushed.

The delivery apparatus 100 is them ready to be inserted into a vessel of a patient during the valve implantation procedure (e.g., using a sheath, such as the sheath 200 of FIG. 3).

In this way, a tray for a packaging for a prosthetic valve delivery apparatus can be configured and used as an integrated shipping/storage and preparation tray for preparing a prosthetic valve and its delivery apparatus for implantation into a patient. As a result, assembly and preparation of the prosthetic valve and delivery apparatus can be performed more easily and consistently in a contained and sterile environment. Specifically, the preparation procedure can be simplified and reduce errors during the procedure. In addition to improving ease of use, this packaging system can make storage and handling of the delivery system easier.

By including a tray with retaining features with snap-like functionality, the delivery system components (such as the shaft of the delivery apparatus) can be dropped into and “snapped” into place inside the tray without having the fold multiple taps or other securing features, thereby increasing the ease of assembly and reducing labor time during manufacturing and packing of the delivery system.

Additionally, by having the curved section of the delivery apparatus shaft that forms the 180-degree bend be in a distal half of the shaft, the curved section is placed in a portion of the shaft that closely matches a patient's anatomy (e.g., curvature in the patient's vasculature occurs closer to the implantation site for the prosthetic valve). Additionally, the curved section forming the 180-degree bend also results in a reduced packaging size of the tray (relative to a straight delivery apparatus shaft without a bend). This can reduce the overall footprint of the tray and delivery system, thereby reducing costs. Further still, by having a recess configured to receive a crimping device disposed in a middle portion of the tray, a stability of the tray 510 when the crimping device is positioned within the recess can be increased.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. A trial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An assembly comprising: a packaging tray comprising a plurality of recesses configured to receive components of a prosthetic valve delivery system, wherein the tray has a length, a first end portion extending along a third of the length, a second end portion, opposite the first end portion, extending along a third of the length, and a middle portion extending along a third of the length from the first end portion to the second end portion; a delivery apparatus disposed within a set of one or more first recesses of the plurality of recesses of the tray such that a shaft of the delivery apparatus has a curved section forming a 180-degree bend along a distal half of the shaft, wherein the curved section is positioned within the first end portion of the tray; and a crimping device configured to be received within a second recess of the plurality of recesses that is positioned within the middle portion of the tray, wherein a valve mounting portion of the delivery apparatus is adjacent the crimping device when the crimping device is positioned within the second recess.

Example 2. The assembly of any example herein, particularly example 1, wherein the curved section of the shaft is positioned entirely within the first end portion of the tray.

Example 3. The assembly of any example herein, particularly either example 1 or example 2, further comprising a strap that extends over the curved section of the shaft and couples to coupling features on a raised surface of the tray such that the curved section is retained within the tray by the strap.

Example 4. The assembly of any example herein, particularly any one of examples 1-3, wherein a handle of the delivery apparatus is positioned entirely within the second end portion of the tray.

Example 5. The assembly of any example herein, particularly example 4, wherein the tray comprises a first side and a second side opposite the first side, the first side and the second side extending along the length of the tray, and wherein the handle is disposed adjacent the first side and the valve mounting portion is disposed adjacent the second side.

Example 6. The assembly of any example herein, particularly any one of examples 1-5, wherein a proximal half of the shaft is positioned within the second end portion of the tray and the middle portion of the tray.

Example 7. The assembly of any example herein, particularly any one of examples 1-6, wherein a proximal half of the shaft is substantially straight.

Example 8. The assembly of any example herein, particularly any one of examples 1-7, wherein at least one first recess of the one or more first recesses comprises a pair of opposing retaining features configured to hold a portion of the shaft within the at least one first recess, the pair of opposing retaining features spaced apart from one another and extending toward one another from opposite sides of the at least one first recess.

Example 9. The assembly of any example herein, particularly any one of examples 1-8, wherein the second recess comprises one or more retaining features extending outward from the second recess, wherein the one or more retaining features are configured to hold the crimping device within the second recess in a specified horizontal and longitudinal orientation when the crimping device is positioned within the second recess.

Example 10. The assembly of any example herein, particularly example 9, wherein the one or more retaining features include protrusions extending outward, in a vertical direction, from a base of the second recess and one or more retaining features extending outward, from a sidewall of the second recess.

Example 11. The assembly of any example herein, particularly any one of examples 1-10, wherein the second recess comprises a rounded, closed end and an open end, and wherein the crimping device extends out the open end and past a side of the tray when the crimping device is positioned within the second recess.

Example 12. The assembly of any example herein, particularly any one of examples 1-11, wherein the plurality of recesses of the tray comprises a third recess, and further comprising a removable fluid bowl disposed in the third recess.

Example 13. The assembly of any example herein, particularly example 12, wherein the removable fluid bowl comprises a bowl portion and holder portion that are foldable relative to one another, wherein the bowl portion is disposed in the third recess and the holder portion is foldable over an open top of the bowl portion, and wherein the holder portion is configured to receive and suspend a prosthetic valve within the bowl portion.

Example 14. The assembly of any example herein, particularly any one of examples 1-13, wherein the plurality of recesses of the tray comprises a fourth recess configured to receive a loader of the prosthetic valve delivery system, and wherein the fourth recess is positioned within the second end portion and the middle portion of the tray.

Example 15. A packaging assembly for storing a delivery system for a prosthetic valve, comprising: a tray comprising a plurality of recesses, wherein the tray comprises a first end portion and a second end portion opposite the first end portion, the first end portion defining a first end of the tray and the second end portion defining a second end of the tray; and a delivery apparatus disposed within a set of one or more first recesses of the plurality of recesses of the tray such that a handle portion of the delivery apparatus is positioned within the first end portion of the tray, a distal half of a shaft of the delivery apparatus has a curved section forming a 180-degree bend positioned within the second end portion of the tray, and a valve mounting portion of the delivery apparatus is positioned within the tray closer to the second end than the first end.

Example 16. The packaging assembly of any example herein, particularly example 15, wherein the handle portion is positioned adjacent a first side of the tray and the valve mounting portion is positioned adjacent a second side of the tray that is opposite the first side, and wherein the first and second sides defined a length of the tray.

Example 17. The packaging assembly of any example herein, particularly example 16, wherein the curved section of the shaft is positioned entirely within the second end portion of the tray and extends along and adjacent to a third side of the tray that extends between the first and second sides.

Example 18. The packaging assembly of any example herein, particularly either example 16 or example 17, wherein a proximal half of the shaft is substantially straight and extends along and adjacent to the first side of the tray.

Example 19. The packaging assembly of any example herein, particularly any one of examples 15-18, further comprising a strap that extends over the curved section of the shaft and couples to coupling features on a raised surface of the tray such that the curved section is retained within the tray by the strap.

Example 20. The packaging assembly of any example herein, particularly any one of examples 15-19, wherein the handle portion of the delivery apparatus is positioned entirely within the first end portion of the tray, and wherein a first recess of the set of one or more first recesses that is configured to receive the handle portion has a length that is longer than the handle portion such that the handle portion can slide within the first recess.

Example 21. The packaging assembly of any example herein, particularly any one of examples 15-20, wherein a proximal half of the shaft is positioned within the first end portion of the tray and a middle portion of the tray that extends between the first end portion and the second end portion.

Example 22. The packaging assembly of any example herein, particularly any one of examples 15-21, wherein at least one first recess of the one or more first recesses comprises a pair of opposing retaining features configured to hold a portion of the shaft within the at least one first recess, the pair of opposing retaining features spaced apart from one another and extending toward one another from opposite sides of the at least one first recess.

Example 23. The packaging assembly of any example herein, particularly any one of examples 15-22, wherein the plurality of recesses comprises a second recess that is configured to receive a crimping device, and wherein the second recess is located within a middle portion of the tray, between the first and second end portions, and adjacent to the valve mounting portion of the delivery apparatus.

Example 24. The packaging assembly of any example herein, particularly example 23, wherein the second recess comprises one or more retaining features extending outward from the second recess, and wherein the one or more retaining features are configured to hold the crimping device within the second recess in a specified horizontal and longitudinal orientation when the crimping device is positioned within the second recess.

Example 25. The packaging assembly of any example herein, particularly example 24, wherein the one or more retaining features include a plurality of protrusions extending outward, in a vertical direction, from a base of the second recess and a pair of retaining features extending outward from opposite sidewalls of the second recess.

Example 26. The packaging assembly of any example herein, particularly any one of examples 15-25, wherein the plurality of recesses of the tray comprises a third recess, and further comprising a removable fluid bowl disposed in the third recess.

Example 27. A packaging assembly for storing a prosthetic valve delivery apparatus, comprising: a tray configured to receive the prosthetic valve delivery apparatus, wherein the tray includes at least one recess; and a removable bowl disposed in the recess, wherein the bowl is configured to receive a liquid.

Example 28. The packaging assembly of any example herein, particularly example 27, wherein the removable bowl comprises a bowl portion and holder portion that are foldable relative to one another, wherein the bowl portion is positioned in the recess and the holder portion is foldable over an open top of the bowl portion, and wherein the holder portion is configured to receive and suspend a prosthetic valve within the bowl portion when folded over the bowl portion.

Example 29. The packaging assembly of any example herein, particularly example 28, wherein the holder portion comprises one or more coupling features at an end of the holder portion that are configured to mate with complementary coupling features of the bowl portion when the holder portion is folded over the bowl portion.

Example 30. The packaging assembly of any example herein, particularly any one of examples 27-29, wherein the removable bowl comprises one or more coupling features that are configured to couple to complementary coupling features disposed in the tray adjacent to the recess.

Example 31. The packaging assembly of any example herein, particularly any one of examples 27-30, wherein the removable bowl comprises one or more mating features that are configured to mate with complementary mating features on a sidewall of the recess.

Example 32. A method comprising sterilizing the prosthetic heart valve, apparatus, and/or assembly of any example.

Example 33. A prosthetic heart valve of any one of examples 1-31, wherein the prosthetic heart valve is sterilized.

The features described herein with regard to any example can be combined with other features described in any one or more of the other examples, unless otherwise stated. For example, any one or more of the features of one packaging assembly can be combined with any one or more features of another packaging assembly. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. An assembly comprising:
a packaging tray comprising a plurality of recesses configured to receive components of a prosthetic valve delivery system, wherein the tray has a length, a first end portion extending along a third of the length, a second end portion, opposite the first end portion, extending along a third of the length, and a middle portion extending along a third of the length from the first end portion to the second end portion;
a delivery apparatus disposed within a set of one or more first recesses of the plurality of recesses of the tray such that a shaft of the delivery apparatus has a curved section forming a 180-degree bend along a distal half of the shaft, wherein the curved section is positioned within the first end portion of the tray; and
a crimping device configured to be received within a second recess of the plurality of recesses that is positioned within the middle portion of the tray, wherein a valve mounting portion of the delivery apparatus is adjacent the crimping device when the crimping device is positioned within the second recess.

2. The assembly of claim 1, wherein the curved section of the shaft is positioned entirely within the first end portion of the tray.

3. The assembly of claim 1, further comprising a strap that extends over the curved section of the shaft and couples to coupling features on a raised surface of the tray such that the curved section is retained within the tray by the strap.

4. The assembly of claim 1, wherein a handle of the delivery apparatus is positioned entirely within the second end portion of the tray.

5. The assembly of claim 4, wherein the tray comprises a first side and a second side opposite the first side, the first side and the second side extending along the length of the tray, and wherein the handle is disposed adjacent the first side and the valve mounting portion is disposed adjacent the second side.

6. The assembly of claim 1, wherein a proximal half of the shaft is positioned within the second end portion of the tray and the middle portion of the tray.

7. The assembly of claim 1, wherein at least one first recess of the one or more first recesses comprises a pair of opposing retaining features configured to hold a portion of the shaft within the at least one first recess, the pair of opposing retaining features spaced apart from one another and extending toward one another from opposite sides of the at least one first recess.

8. The assembly of claim 1, wherein the second recess comprises one or more retaining features extending outward from the second recess, wherein the one or more retaining features are configured to hold the crimping device within the second recess in a specified horizontal and longitudinal orientation when the crimping device is positioned within the second recess.

9. The assembly of claim 8, wherein the one or more retaining features include protrusions extending outward, in a vertical direction, from a base of the second recess and one or more retaining features extending outward, from a sidewall of the second recess.

10. A packaging assembly for storing a delivery system for a prosthetic valve, comprising:
a tray comprising a plurality of recesses, wherein the tray comprises a first end portion and a second end portion opposite the first end portion, the first end portion defining a first end of the tray and the second end portion defining a second end of the tray; and
a delivery apparatus disposed within a set of one or more first recesses of the plurality of recesses of the tray such that a handle portion of the delivery apparatus is positioned within the first end portion of the tray, a distal half of a shaft of the delivery apparatus has a curved section forming a 180-degree bend positioned within the second end portion of the tray, and a valve mounting portion of the delivery apparatus is positioned within the tray closer to the second end than the first end.

11. The packaging assembly of claim 10, wherein the handle portion is positioned adjacent a first side of the tray and the valve mounting portion is positioned adjacent a second side of the tray that is opposite the first side, and wherein the first and second sides define a length of the tray.

12. The packaging assembly of claim 11, wherein the curved section of the shaft is positioned entirely within the second end portion of the tray and extends along and adjacent to the second end of the tray that extends between the first and second sides.

13. The packaging assembly of claim 11, wherein a proximal half of the shaft is substantially straight and extends along and adjacent to the first side of the tray.

14. The packaging assembly of claim 10, wherein the handle portion of the delivery apparatus is positioned entirely within the first end portion of the tray, and wherein a first recess of the set of one or more first recesses that is configured to receive the handle portion has a length that is longer than the handle portion such that the handle portion can slide within the first recess.

15. The packaging assembly of claim 10, wherein a proximal half of the shaft is positioned within the first end portion of the tray and a middle portion of the tray that extends between the first end portion and the second end portion.

16. A packaging assembly for storing a prosthetic valve delivery apparatus, comprising:
a tray configured to receive the prosthetic valve delivery apparatus, wherein the tray includes at least one recess; and
a removable bowl disposed in the recess, wherein the bowl is configured to receive a liquid.

17. The packaging assembly of claim 16, wherein the removable bowl comprises a bowl portion and holder portion that are foldable relative to one another, wherein the bowl portion is positioned in the recess and the holder portion is foldable over an open top of the bowl portion, and wherein the holder portion is configured to receive and suspend a prosthetic valve within the bowl portion when folded over the bowl portion.

18. The packaging assembly of claim 17, wherein the holder portion comprises one or more coupling features at an end of the holder portion that are configured to mate with complementary coupling features of the bowl portion when the holder portion is folded over the bowl portion.

19. The packaging assembly of claim 16, wherein the removable bowl comprises one or more coupling features that are configured to couple to complementary coupling features disposed in the tray adjacent to the recess.

20. The packaging assembly of claim 16, wherein the removable bowl comprises one or more mating features that are configured to mate with complementary mating features on a sidewall of the recess.
